(19) United States
(12) Patent Application Publication
(10) Pub. No.: US 2026/0199082 A1
(43) Pub. Date: Jul. 16, 2026

(54) TEXTILES, METHODS OF MAKING THE SAME AND IMPLANTABLE MEDICAL DEVICES USING THE SAME

(71) Applicant: Edwards Lifesciences Corporation, Irvine, CA (US)

(72) Inventors: Rupesh Gajanan Nawalakhe, Trabuco Canyon, CA (US); Sandip Vasant Pawar, Irvine, CA (US)

(21) Appl. No.: 19/565,369

(22) Filed: Mar. 12, 2026

Related U.S. Application Data
(63) Continuation of application No. PCT/US2024/046543, filed on Sep. 13, 2024.
(60) Provisional application No. 63/582,834, filed on Sep. 14, 2023.

(51) Int. Cl. A61F 2/24

(52) U.S. Cl. CPC A61F 2/2415; A61F 2/2418; A61F 2210/0071; A61F 2210/0076; A61F 2240/001; A61F 2250/0017; A61F 2250/0031; A61F 2250/0036

(57) ABSTRACT

Disclosed herein is a method of forming coverings for implantable devices that improve PVL and can function as a frictional element. Also described herein are prosthetic valves having the formed coverings.

### CROSS-REFERENCE TO RELATED APPLICATIONS

This application is a continuation of International Application No. PCT/US2024/046543, filed September 13, 2024, claims the benefit of U.S. Patent Application No. 63/582,834 filed September 14, 2023, the content of which is incorporated herein by reference in its entirety.

### FIELD

The present application concerns methods of making textile materials that can be used on implantable prosthetic valves. The present application also concerns implantable medical devices comprising such textile materials.

### BACKGROUND

The heart can suffer from various valvular diseases or malformations that result in significant malfunctioning of the heart and ultimately require the replacement of the native heart valve with an artificial valve. Human heart valves, which include the aortic, pulmonary, mitral, and tricuspid valves, function essentially as one-way valves operating in synchronization with the pumping heart. The valves allow blood to flow downstream but block blood from flowing upstream. Diseased heart valves exhibit impairments such as narrowing of the valve or regurgitation, which inhibits the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open-heart surgery. Replacement valves are being designed to be delivered through minimally invasive procedures and even percutaneous procedures. Such replacement valves often include a tissue-based valve body that is connected to an expandable frame that is then delivered to the native valve's annulus.

These replacement valves are often intended to at least partially block blood flow. However, a problem occurs when blood flows around the valve on the outside of the prosthesis. For example, in the context of replacement heart valves, paravalvular leakage has proven particularly challenging. An additional challenge relates to the ability of such prostheses to be secured relative to intra-luminal tissue, e.g., tissue within any body lumen or cavity, in an atraumatic manner. Further challenges arise when trying to controllably deliver and secure such prostheses in a location such as at a native mitral valve. These replacement valves are often intended to at least partially block blood flow.

Because of the drawbacks associated with conventional open-heart surgery, percutaneous and minimally-invasive surgical approaches are garnering intense attention. In one technique, a prosthetic valve is configured to be implanted in a much less invasive procedure by way of catheterization. For instance, U.S. Pat. Nos. 5,411,522 and 6,730,118, which are incorporated herein by reference, describe collapsible transcatheter heart valves that can be percutaneously introduced in a compressed state on a catheter and expanded in the desired position by balloon inflation or by utilization of a self-expanding frame or stent. In yet another example, U.S. U.S. Publication Nos. 2014/0277390, 2014/0277422, 2014/0277427, 2015/0328000, and 2019/0328515, which are incorporated herein by reference in their entireties, describe heart valve prostheses for replacing a native mitral valve, including a self-expanding frame with a plurality of anchoring members that are designed be deployed within a body cavity and prevent axial flow of fluid around an exterior of the prosthesis.

However, problems still can occur. For example, in the context of replacement heart valves, paravalvular leakage has proven particularly challenging.

An additional challenge relates to the ability of such prostheses to be secured relative to intra-luminal tissue, e.g., tissue within any body lumen or cavity, in an atraumatic manner. Further challenges arise when trying to controllably deliver and secure such prostheses in a location such as at a native mitral valve.

These needs and others are at least partially satisfied by the present disclosure.

### SUMMARY

Some of the aspects of the present disclosure relate to methods of making prosthetic devices comprising various textiles. In some aspects, disclosed herein are methods forming an implantable prosthetic valve, wherein the methods comprise: (a) forming a valve covering material, wherein the step of forming comprises: (i) weaving a double cloth comprising a top portion and a bottom portion, wherein the top portion and bottom portion are spaced at a first length; wherein the top portion comprises a first woven base layer having a first surface and an opposing second surface and comprising a first plurality of warp yarns and a first plurality of weft yarns; wherein the bottom portion comprises a second woven base layer having a first surface and an opposing second surface comprising a second plurality of warp yarns and a second plurality of weft yarns; wherein the top portion and bottom portion are coupled with a plurality of pile yarns such that the second surface of the first woven base layer faces the second surface of the second woven base layer, wherein the plurality of pile yarns extend between the second surface of the first woven base layer and the second surface of the second woven base layer along the first length; wherein a first portion of the plurality of pile yarns is interwoven with the first woven base layer and a second portion of the plurality of pile yarns is interwoven with the second woven base layer; (ii) cutting through the plurality of pile yarns transversely to the first length to form: a first cloth comprising the first woven base layer having a first predetermined pattern and a first cut pile layer comprising a portion of the plurality of pile yarns and extending outwardly from the second surface of the first woven base layer, wherein the first cut pile layer has a second predetermined pattern and a first predetermined thickness; a second cloth material comprising the second woven base layer having the first predetermined pattern and a second cut pile layer comprising a portion of the plurality of pile yarns and extending outwardly from the second surface of the second woven base layer, wherein the second cut pile layer has the second predetermined pattern and a second predetermined thickness; and wherein at least a portion of the first portion of the plurality of pile yarns is integrally fused with at least a portion of the first woven base layer; and/or wherein at least a portion of the second portion of the plurality of pile yarns is integrally fused with at least a portion of the second woven base layer; and (b) positioning at least one of the first and/or second cloth materials on a prosthetic valve frame to form the valve covering such that at least a portion of the first and/or second cut pile layer is exposed to a natural anatomy.

Also disclosed herein are methods of forming an implantable prosthetic valve, wherein the methods comprise: (a) forming a valve covering material, wherein the step of forming comprises: weaving a double cloth comprising a top portion and a bottom portion, wherein the top portion and bottom portion are spaced at a first length; wherein the top portion comprises a first woven base layer having a first surface and an opposing second surface and comprising a first plurality of warp yarns and a first plurality of weft yarns; wherein the bottom portion comprises a second woven base layer having a first surface and an opposing second surface comprising a second plurality of warp yarns and a second plurality of weft yarns; wherein the first and second woven base layers have a first predetermined pattern; wherein the top portion and bottom portion are coupled with a plurality of pile yarns such that the second surface of the first woven base layer faces the second surface of the second woven base layer, wherein the plurality of pile yarns extend between the second surface of the first woven base layer and the second surface of the second woven base layer along the first length; wherein a first portion of the plurality of pile yarns is interwoven with the first woven base layer and a second portion of the plurality of pile yarns is interwoven with the second woven base layer; wherein the first woven base layer is bio-resorbable; wherein at least a portion of the second portion of the plurality of pile yarns is integrally fused with at least a portion of the second woven base layer; and (b) positioning the covering on a prosthetic valve frame to form the valve covering such that the first surface of the first woven base layer.

Also disclosed herein is a prosthetic valve comprising: at least one covering comprising: a woven base layer having a first surface and a second surface, and a first predetermined pattern; a cut pile layer, wherein the cut pile layer has a predetermined thickness and a second predetermined pattern; and wherein the cut pile layer comprises a plurality of yarns outwardly extending from the second surface of the woven base layer, wherein at least one yarn of the plurality of yarns present in the cut pile layer is a multifilament yarn or a monofilament yarn, and wherein at least a portion of the plurality of pile yarns is integrally fused with the woven base layer; and wherein the at least one covering is positioned on a valve frame, such that the first surface of the woven base layer is adjacent to the frame.

Still further disclosed herein is a prosthetic valve comprising: at least one covering comprising: a double cloth material comprising a top portion and a bottom portion, wherein the top portion and bottom portion are spaced at a first length; wherein the top portion comprises a first woven base layer having a first surface and an opposing second surface and comprising a first plurality of warp yarns and a first plurality of weft yarns; wherein the bottom portion comprises a second woven base layer having a first surface and an opposing second surface comprising a second plurality of warp yarns and a second plurality of weft yarns; wherein the first and second woven base layers have a first predetermined pattern; wherein the top portion and bottom portion are coupled with a plurality of pile yarns such that the second surface of the first woven base layer faces the second surface of the second woven base layer, wherein the plurality of pile yarns extend between the second surface the first woven base layer and the second surface of the second woven base layer along the first length in a second predetermined pattern; wherein a first portion of the plurality of pile yarns is interwoven with the first woven base layer and a second portion of the plurality of pile yarns is interwoven with the second woven base layer; wherein the first woven base layer is bio-resorbable; wherein at least a portion of the second portion of the plurality of pile yarns is integrally fused with at least a portion of the second woven base layer; and wherein the at least one covering is positioned on a valve frame such that the second woven base layer is adjacent to the valve frame and the first woven base layer is exposed to a native anatomy.

Additional aspects of the disclosure will be set forth, in part, in the detailed description, figures, and claims that follow, and in part, will be derived from the detailed description or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

FIGS. 1A–AC show an exemplary double cloth: FIG. 1A shows a top view of the woven fabric; FIG. 1B shows a cross-sectional view of an exemplary double cloth and FIG. 1C shows the cutting step of the exemplary double cloth to form exemplary first and second clothes.

FIG. 2 shows an exemplary cloth formed after the step of cutting.

FIGS. 3A–3D show photographs of an exemplary cloth. FIG. 3A shows a double cloth material where the plurality of pile yarns comprises multifilament fibers. FIG. 3B shows the cloth material with the cut pile layer. FIG. 3C shows a double cloth material where the plurality of pile yarns comprises monofilament fiber. FIG. 3D shows the cloth material with the cut pile layer.

FIG. 4A shows an exemplary prosthetic valve with exemplary cloth materials according to one aspect of the disclosure.

FIG. 4B shows an exemplary prosthetic valve with exemplary cloth materials according to another aspect of the disclosure.

FIGS. 5A–5C show various patterns of the pile yarn.

FIG. 6A shows an exemplary cloth according to one aspect of the disclosure.

FIG. 6B shows an exemplary cloth according to another aspect of the disclosure.

FIG. 7 shows an exemplary method of making an exemplary cloth in one aspect.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

### DEFINITIONS

As used in this application and in the claims, the singular forms “a,” “an,” and “the” include the plural forms unless the context clearly dictates otherwise. Thus, for example, reference to a “yarn” includes aspects having two or more such yarns unless the context clearly indicates otherwise.

It is also to be understood that the terminology used herein is for the purpose of describing particular aspects only and is not intended to be limiting. As used in the specification and in the claims, the term “comprising” can include the aspects “consisting of” and “consisting essentially of.” Additionally, the term “includes” means “comprises.”

For the terms “for example,” “exemplary,” and “such as,” and grammatical equivalences thereof, the phrase “and without limitation” is understood to follow unless explicitly stated otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint and independently of the other endpoint. Unless stated otherwise, the term “about” means within 5% (e.g., within 2% or 1%) of the particular value modified by the term “about.” It is further understood that if the value is disclosed as “about 1,” then the value of “1” is also disclosed.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the terms “optional” or “optionally” mean that the subsequently described event or circumstance can or cannot occur and that the description includes instances where said event or circumstance occurs and instances where it does not.

Further, the terms “coupled” and “associated” generally mean electrically, electromagnetically, and/or physically (e.g., mechanically or chemically) coupled or linked and do not exclude the presence of intermediate elements between the coupled or associated items.

It will be understood that when an element is referred to as being "connected" or "coupled" to another element, it can be directly connected or coupled to the other element, or intervening elements can be present. In contrast, when an element is referred to as being "directly connected" or "directly coupled" to another element, there are no intervening elements present. Other words used to describe the relationship between elements or layers should be interpreted in a like fashion (e.g., "between" versus "directly between," "adjacent" versus "directly adjacent," "on" versus "directly on").

It will be understood that although the terms "first," "second," etc., can be used herein to describe various elements, components, regions, layers and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example aspects.

Spatially relative terms, such as,“ "beneath," "below," "lower," "above," "upper," “upward,” “downward,” “top,” “bottom,” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as "below" or "beneath" other elements or features would then be oriented "above" the other elements or features. Thus, the term "below" can encompass both an orientation of above and below. The device can be otherwise oriented (rotated 90 degrees or at other orientations), and the spatially relative descriptors used herein are interpreted accordingly.

Terms such as “proximal,” “distal,” “radially outward,” “radially inward,” “outer,” “inner,” and “side” describe the orientation and/or location of portions of the components or elements within a consistent but arbitrary frame of reference which is made clear by reference to the text and the associated drawings describing the components or elements under discussion. Such terminology can include the words specifically mentioned above, derivatives thereof, and words of similar import.

As used herein, the term "and/or" includes any and all combinations of one or more of the associated listed items. It is also understood that the term “and/or” includes where one or another of the associated listed items is present, and the aspects where both of the associated listed items are present or any combinations of the associated listed items are present.

The terms “fiber” or “filament” can be used interchangeably. The terms “fiber” (or “filament”) and “material comprising a plurality of fibers (filaments)” are used herein according to their broad and ordinary meanings and can refer to any type of natural or synthetic substance or material that is significantly longer than it is wide, including any elongate or relatively fine, slender, and/or threadlike piece, filament, cord, yarn, plie, strand, line, string, or portion thereof. Furthermore, “fiber” or “material comprising a plurality of fibers” can refer to a single filament or collectively to a plurality of filaments. Examples of material comprising a plurality of fibers in accordance with aspects of the present disclosure include, but are not limited to, any type of cloth, fabric, or textile. It is understood that in certain unlimiting aspects, the term “material comprising a plurality of fibers” can refer to cloth, fabric, textile, or interlocking-fiber material that can cover or form certain features of the disclosed devices.

The term “fiber,” as used herein, includes fibers of extreme or indefinite length (e.g., filaments) and fibers of short length (e.g., staple fibers). It is further understood that unless specifically stated otherwise, the filaments can be monofilaments, multifilaments, braided filaments, unbraided filaments, textured filaments, untextured filaments, and the like.

As used herein, the term "substantially" means that the subsequently described event or circumstance completely occurs or that the subsequently described event or circumstance generally, typically, or approximately occurs.

Still further, the term “substantially” can, in some aspects, refer to at least about 90 %, at least about 91 %, at least about 92 %, at least about 93 %, at least about 94 %, at least about 95 %, at least about 96 %, at least about 97 %, at least about 98 %, at least about 99 %, or about 100 % of the stated property, component, composition, or other condition for which substantially is used to characterize or otherwise quantify an amount.

As used herein, the term “substantially,” in, for example, the context “substantially identical” or “substantially similar,” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may, in some cases, be rearranged or performed concurrently. Further, descriptions and disclosures provided in association with one particular aspect are not limited to that aspect and may be applied to any aspect disclosed.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

### METHODS

The present disclosure is directed to methods of forming an implantable prosthetic valve comprising a covering that is configured to address current issues associated with paravalvular leakage, to tune tissue ingrowth as desired, to improve the biocompatibility of valve, and, if needed, to form a valve having a fictional element that would assist in stabilizing the valve in the natural anatomy of the patient.

### CLOTH

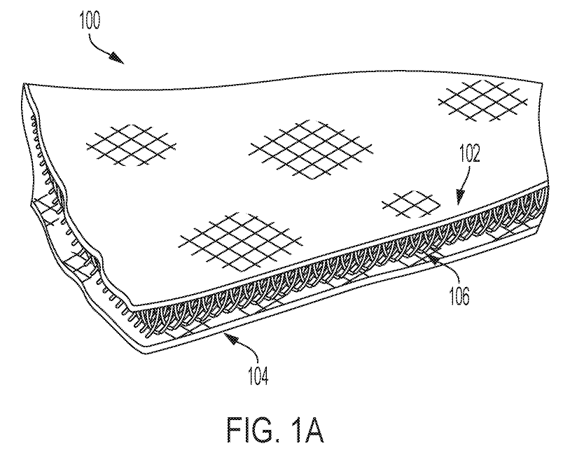

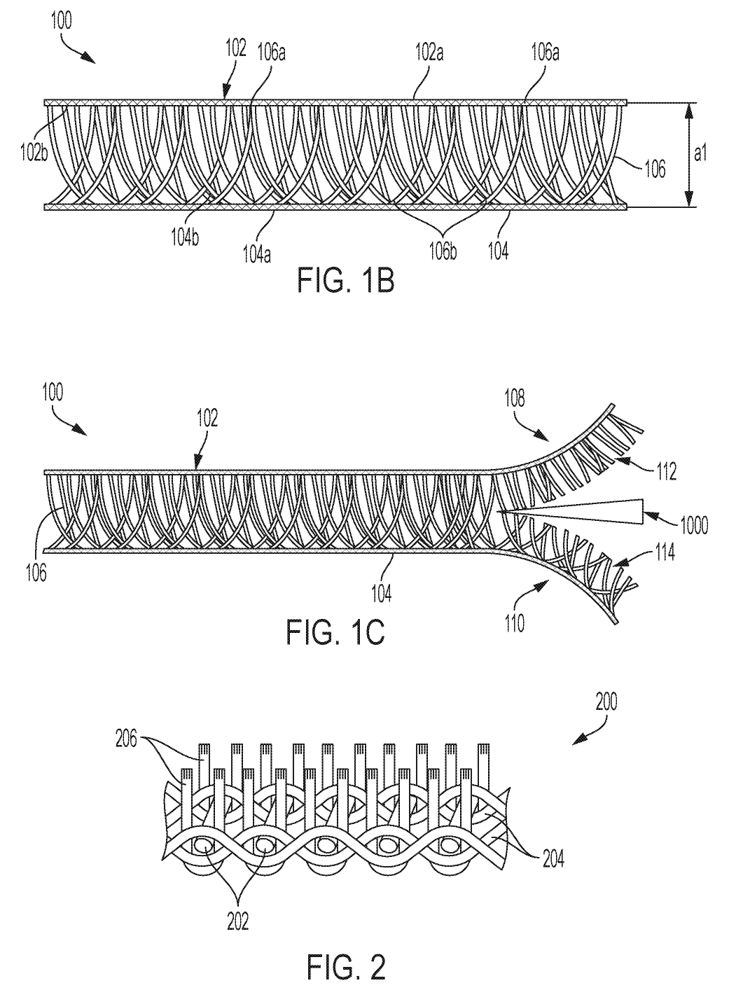

Disclosed herein is a method of forming an implantable prosthetic device comprising forming a valve covering. In such aspects, the step of forming the valve covering comprises weaving a double cloth comprising a top portion and a bottom portion. Exemplary double cloth is shown in FIGS. 1A–1B. For example, the top portion and bottom portion can be spaced apart at a first length a₁. It is understood, however, that the terms “top portion” and “bottom portion” are only used to show the spatial relationship between the two portions, and the top portion can become the bottom portion if the cloth is turned around. In still further aspects, the top portion comprises a first woven base layer 102 having a first surface 102a and an opposing second surface 102b. The first woven base layer 102 comprises a first plurality of warp yarns and a first plurality of weft yarns. In still further aspects, the bottom portion comprises a second woven base layer 104 having a first surface 104a and an opposing second surface 104b. The second woven base layer 104 comprises a second plurality of warp yarns and a second plurality of weft yarns. In still further aspects, the top portion and bottom portion are coupled with a plurality of pile yarns 106 such that the second surface 102b of the first woven base layer 102 faces the second surface 104b of the second woven base layer 104. In still further aspects, the plurality of pile yarns 106 extend between the second surface of the first woven base layer and the second surface of the second woven base layer along the first length a₁.

In still further aspects, the double cloth disclosed herein is woven such that a first portion (as shown in some locations as 106a) of the plurality of pile yarns 106 is interwoven with the first woven base layer 102 and a second portion (as shown in some locations as 106b) of the plurality of pile yarns 106 is interwoven with the second woven base layer 104.

In still further aspects, and as shown in FIG. 1C, the method comprises cutting 1000 through the plurality of pile yarns 106 transversely to the first length a₁. It is understood that cutting 1000 can be done by any known in the art method, for example, the cutting can be done by a blade (stationary or moving), a laser, a saw, or any combination thereof.

When the double cloth is cut through the plurality of pile yarns, two separate cloths are formed. In such aspects, a first cloth material 108 comprises the first woven base layer 102 having a first predetermined pattern and a first cut pile layer 112. In such aspects, the first cut pile layer 112 comprises a portion of the plurality of pile yarns106 that is left with the first woven base layer 102 after the cutting step. In such aspects, the portion of the plurality of yarn extends outwardly from the second surface 102b of the first woven base layer. In such exemplary and unlimiting aspects, the first cut pile layer 112 has a second predetermined pattern and a first predetermined thickness. At the same time, a second cloth material 110 is also formed. In such aspects, the second cloth material 110 comprises the second woven base layer 104 having the first predetermined pattern and a second cut pile layer 114 comprising a portion of the plurality of pile yarns106 that is left with the second woven base layer 104 after the cutting step. In such aspects, the portion of the plurality of yarn extends outwardly from the second surface 104b of the second woven base layer 104. In still further aspects, the second cut pile layer 114 has a second predetermined pattern and a second predetermined thickness.

It is understood that the first predetermined thickness and the second predetermined thickness can be defined by a height of the cut pile layer. In still further aspects, the first and/or second woven base layers can have a thickness. It is understood that such thickness can be determined by the type of fiber used in woven procedures and can be, for example, of about 0.06 mm to about 0.15 mm, including exemplary values of about 0.07 mm, about 0.08 mm, about 0.09 mm, about 0.1 mm, about 0.11 mm, about 0.12 mm, about 0.13 mm, and about 0.14 mm. In still further aspects, the thickness can have any value between any two foregoing values or within a range formed by any two foregoing values. For example, and without limitations, the thickness can be about 0.06 mm to about 0.14 mm, about 0.06 mm to about 0.13 mm, about 0.06 mm to about 0.12 mm, about 0.06 mm to about 0.11 mm, about 0.06 mm to about 0.10 mm, about 0.06 mm to about 0.09 mm, about 0.06 mm to about 0.08 mm, about 0.08 mm to about 0.15 mm, about 0.09 mm to about 0.15 mm, about 0.10 mm to about 0.15 mm, about 0.11 mm to about 0.15 mm, about 0.12 mm to about 0.15 mm, about 0.13 mm to about 0.15 mm, and so on.

In still further aspects, the covering is constructed such that at least a portion of the first portion of the plurality of pile yarns is integrally fused with at least a portion of the first woven base layer; and/or wherein at least a portion of the second portion of the plurality of pile yarns is integrally fused with at least a portion of the second woven base layer.

In still further aspects, the methods comprise a step of positioning at least one of the first and/or second cloth materials on a prosthetic valve frame to form the valve covering such that at least a portion of the first and/or second cut pile layer is exposed to natural anatomy. It is understood, however, that either or both of the formed cloth can be used in any of the desired applications. In certain aspects, one cloth can be sacrificed to form another cloth of the desired pattern and cut pile yarn thickness as needed and, as discussed below.

In still further aspects, the first length can be determined depending on the desired application and can be anywhere between about 0.5 mm and about 10 mm, including exemplary values of about 0.6 mm, about 0.7 mm, about 0.8 mm, about 1 mm, about 1.2 mm, about 1.5 mm, about 1.7 mm, about 2 mm, about 2.2 mm, about 2.5mm, about 2.7 mm, about 3 mm, about 3.2 mm, about 3.5 mm, about 3.7 mm, about 4 mm, about 4.2 mm, about 4.5 mm, about 4.7 mm, about 5 mm, about 5.2 mm, about 5.5 mm, about 5.7 mm, about 6 mm, about 6.2 mm, about 6.5 mm, about 6.7 mm, about 7 mm, about 7.2 mm, about 7.5 mm, about 7.7 mm, about 8 mm, about 8.2 mm, about 8.5 mm, about 8.7 mm, about 9 mm, about 9.2 mm, about 9.5 mm, and about 9.7 mm. It is understood that the first length can have any value that falls within any two foregoing values or falls within any range formed by any two foregoing values. For example, the first length can be about 0.5 mm to about 8 mm, 0.5 mm to about 5 mm, 0.5 mm to about 3 mm, 0.5 mm to about 1 mm, 1 mm to about 10 mm, 3 mm to about 10 mm, 5 mm to about 10 mm, 8 mm to about 10 mm, and so on.

In still further aspects, the first predetermined thickness of the first cloth material and the second predetermined thickness of the second cloth material can be defined by where the plurality of pile yarns are cut through. For example, in some aspects, the first and the second predetermined thicknesses are substantially the same. In such aspects, the plurality of pile yarns are cut in the middle. In yet still further aspects, the first predetermined thickness can be larger than the second predetermined thickness. In yet still further aspects, the first predetermined thickness can be smaller than the second predetermined thickness.

In certain aspects, the first and/or second predetermined thickness can be from about 0.1 mm to about 5 mm, including exemplary values of about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, and about 4.9 mm. It is understood that, for example, in some aspects, it would be desirable to obtain the first or the second predetermined thickness of about 0.1 mm to less than about 1 mm, including exemplary values of about 0.2 mm, about 0.3 mm, about 0.4 mm, about 0.5 mm, about 0.6 mm, about 0.7 mm, about 0.8 mm, and about 0.9 mm. It is understood that the first and/or second predetermined thickness can have any value that falls within any two foregoing values or falls within any range formed by any two foregoing values. For example, the first and/or second predetermined thickness can be about 0.1 mm to about 4 mm, 0.1 mm to about 3 mm, 0.1 mm to about 2 mm, 0.1 mm to about 1 mm, 0.1 mm to about 0.5 mm, 0.5 mm to about 5 mm, 1 mm to about 5 mm, 2 mm to about 5 mm, 3 mm to about 5 mm, and so on.

In such exemplary aspects, one of the cloth materials can be sacrificed or used for a different purpose.

In still further aspects, the first predetermined pattern of the first and the second woven base layer comprises a plain 1:1 pattern, warp rib 2/1, warp rib 2/2, plain weave, and its derivatives or any combination thereof. It is understood that in double cloth materials, the first and the second woven layers will have the same first predetermined pattern.

### WARP YARNS

In still further aspects, the first plurality of warp yarns and the second plurality of warp yarns comprise the same material. Yet, in other aspects, the first plurality of warp yarn can comprise material that is different from the second plurality of warp yarns.

In certain aspects, the first plurality of warp yarns can comprise multifilament yarns. Yet, in other aspects, the first plurality of warp yarns can comprise monofilament yarns. In still further aspects, the first plurality of warp yarns can comprise a combination of yarns that some are multifilament, and some are monofilament yarns. In certain aspects, the second plurality of warp yarns can comprise multifilament yarns. Yet, in other aspects, the second plurality of warp yarns can comprise monofilament yarns. In still further aspects, the second plurality of warp yarns can comprise a combination of yarns that some are multifilament, and some are monofilament yarns.

It is understood that also disclosed herein are aspects where the first plurality of warp yarns is monofilament while the second plurality of warp yarns is multifilament. Yet also disclosed are aspects in which the first plurality of warp yarns is multifilament and the second plurality of warp yarns is monofilament. Also disclosed herein are the aspects where the first plurality of warp yarns comprises both monofilament and multifilament yarns, while the second plurality of warp yarns is either monofilament or multifilament. Similarly disclosed are aspects where the second plurality of warp yarns comprises both monofilament and multifilament yarns, while the first plurality of warp yarns is either monofilament or multifilament. Further disclosed are aspects in which the first and second pluriities of warp yarns are the same.

In still further aspects, the first plurality of warp yarns can comprise a number of filaments that are different from the second plurality of warp yarns. It is understood, however, that also disclosed herein are aspects where each warp yarn of the first plurality of warp yarns and/or each yarn of the second plurality of warp yarns can comprise the same number of filaments or a different number of filaments. In still further aspects, the first plurality of warp yarns and/or the second plurality of warp yarns can comprise any number of filaments, for example, it can comprise about 1 to about 200 filaments, including exemplary values of about 2, about 5, about 8, about 10, about 20, about 30, about 40, about 50, about 60, about 70, about 80, about 90, about 100, about 110, about 120, about 130, about 140, about 150, about 160, about 170, about 180, and about 190. It is understood that these values are only exemplary, and the disclosed herein warp yarns can comprise any number of filaments that fall between any two mentioned above values or that fall in any range formed by any two foregoing values. For example, and without limitations, the yarns in the first plurality of warp yarns and/or the second plurality of warp yarns can also comprise about 7 filaments, about 55 filaments, about 115 filaments, and the like. In other words, the yarns in the first plurality of warp yarns and/or second plurality of warp yarns comprise about 1 to 15 filaments, about 10 to about 100 filaments, or about 20 filaments to about 200 filaments, and the like.

In still further aspects, the first plurality of warp yarns can comprise one or more filaments having the same or different deniers. Yet in still further aspects, each yarn in the first plurality of warp yarns can have filaments having the same denier. While in other aspects, each yarn in the first plurality of warp yarns can have filaments having a different denier. Yet still, in further aspects, the first plurality of warp yarns can comprise yarns that have filaments with the same and/or different deniers.

In still further aspects, the second plurality of warp yarns can comprise one or more filaments having the same or different deniers. Yet in still further aspects, each yarn in the second plurality of warp yarns can have filaments having the same denier. While in other aspects, each yarn in the second plurality of warp yarns can have filaments having a different denier. Yet still, in further aspects, the second plurality of warp yarns can comprise yarns that have filaments with the same and/or different deniers.

In still further aspects, at least a portion of the first plurality of warp yarns has the same or different denier as at least a portion of the second plurality of warp yarns.

In still further aspects, at least one yarn of the first and/or second plurality of warp yarns has a denier of about 15 D to about 50 D, including exemplary values of about 20 D, about 25 D, about 30 D, about 35 D, about 40 D, and about 45 D. It is understood that at least one yarn of the first and/or second plurality of warp yarns can have any denier that falls between any two foregoing values or within any range formed by any two foregoing values. For example, at least one yarn of the first and/or second plurality of warp yarns can have denier of about 15D to about 45 D, about 15D to about 40 D, about 15D to about 35 D, about 15D to about 30 D, about 15D to about 25 D, about 15D to about 20 D, about 20 D to about 50 D, about 25 D to about 50 D, about 30 D to about 50 D, about 35 D to about 50 D, about 40 D to about 50 D, about 45 D to about 50 D, and so on.

In still further aspects, the filaments present in the first and/or second plurality of warp yarns can have any diameter suitable for the desired application. In certain aspects, the filament can have a diameter from about 1 μm to about 25 μm, including exemplary values of about 2 μm, about 5 μm, about 7 μm, about 10 μm, about 12 μm, about 15 μm, about 17 μm, about 20 μm, and about 22 μm. It is understood that the filament can have any diameter between any two foregoing values or in any range formed by any two foregoing values. For example, and without limitation, the filament can have a diameter from about 3 μm to about 8 μm, or from about 11μm to about 22 μm, or from about 15 μm to about 25 μm.

It is understood that in order to keep a desirable overall profile of the skirt (relatively low overall profile) for transcatheter valve implantation, it could be desirable to choose a thinner filament for the plurality of warp yarns. In such aspects, the base layer can have a lower profile while maximizing the use of the pile cut for sealing purposes.

In still further aspects, at least one of the first and/or second plurality of warp yarns comprise a twisted yarn, a textured yarn, a flat, or any combination thereof.

It is understood that if the at least one of the first and/or second plurality of warp yarns comprises the textured yarn, such a textured yarn can be prepared by any known in the art methods. In certain exemplary and unlimiting aspects, the textured yarn can be selected from a friction-textured yarn, a pin-textured yarn, an air-textured yarn, a belt-textured yarn, a stuffer box-textured yarn, or any combination thereof. Still, in further exemplary and unlimiting aspects, the textured yarn can be produced by friction texturing, or pin or false twist texturing, air texturing, stuffer box texturing, or any combination thereof. In still further exemplary aspects, the textured yarn is a friction-textured yarn. It is further understood that various textured yarns can have various degrees of texturizing. The degree of texturing is evaluated by measuring crimp contraction, stretch potential, and bulk ratio of the textured yarn. It is understood that also disclosed are aspects where the warp yarn does not comprise a textured yarn if a high surface area of the yarn is not desired.

In certain aspects, at least one yarn in the first and/or second plurality of warp yarns comprises an elastomeric material. In such exemplary and unlimiting aspects, the elastomeric material can comprise one or more thermoplastic polyurethanes, elastomeric polyolefins, silicon-based materials, copolymers thereof, or any combination thereof.

While in still further aspects, at least one yarn in the first and/or second plurality of warp yarns comprises at least one low melt polymer. For example, and without limitations, the at least one low-melt polymer can comprise one or more of low-melt polyester (or a copolymer thereof), polypropylene, thermoplastic polyurethane, polyamide (Nylon 6, Nylon 6,6, Nylon 12, etc.), and the like and any combination thereof.

In certain aspects, yarns in the first and/or second plurality of warp yarns can also comprise a composite filament. It is understood that, as used herein, the composite filament relates to a filament that can comprise one or more different materials. In certain aspects, the composite filament is a bi-component filament that can have any configuration, for example, and without limitation, a side-by-side configuration, a core/sheath configuration, a segmented configuration, an islands-in-the-sea configuration, or any combination thereof.

Yet, in still further aspects, the one or more bi-component filaments can comprise the core/sheath configuration. In such exemplary and unlimiting aspects, a sheath can comprise a polymer with a lower melting temperature than a polymer in a core.

It is further understood that the aspects described herein comprise yarns that are biocompatible. Also disclosed herein are aspects that comprise yarns comprising permanent implant-grade polymers. In still further aspects, at least one yarn of the first and/or second plurality of warp yarns can comprise one or more filaments comprising one or more non-resorbable materials. In such exemplary and unlimiting aspects, wherein the one or more non-resorbable materials comprise polyester, co-polyesters, polyamide, polyolefin, polyurethanes, natural fibers, fluoropolymers, polyethers, polyureas, copolymers thereof, metal wires, or any combination thereof.

In still further aspects, the at least one yarn of the first and/or second plurality of warp yarns comprise PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, cobalt-chromium (Co-Cr), nitinol (NiT), stainless steel, or a combination thereof.

In still further aspects, at least one yarn of the first and/or second plurality of warp yarns comprise one or more filaments comprising one or more bio-resorbable materials. In such exemplary and unlimiting aspects, the one or more bio-resorbable materials comprise PEO, PGA, PLLA, or a combination thereof.

In still further aspects, at least one yarn of the first and/or second plurality of warp yarn is a twisted yarn with about 3 to about 16 turns per inch of yarn, including exemplary values of about 4, about 5, about 6, about 7, about 8, about 9, about 10, about 11, about 12, about 13, about 14, and about 15 turns per inch of yarn. It is understood that the at least one yarn of the first and/or second plurality of warp yarn can have a twist having any value between any two foregoing values or can fall within any range formed by any two foregoing values. For example, and without limitations, the at least one yarn of the first and/or second plurality of warp yarn is a twisted yarn with about 3 to about 15 turns per inch of yarn, about 3 to about 12 turns per inch of yarn, about 3 to about 10 turns per inch of yarn, about 3 to about 8 turns per inch of yarn, about 3 to about 5 turns per inch of yarn, about 5 to about 16 turns per inch of yarn, about 8 to about 16 turns per inch of yarn, about 10 to about 16 turns per inch of yarn, about 12 to about 16 turns per inch of yarn, about 15 to about 16 turns per inch of yarn, and so on.

### WEFT YARN

In still further aspects, the first plurality of weft yarns and the second plurality of weft yarns comprise the same material. Yet, in other aspects, the first plurality of weft yarn can comprise material that is different from the second plurality of weft yarns.

In certain aspects, the first plurality of weft yarns can comprise multifilament yarns. Yet, in other aspects, the first plurality of weft yarns can comprise monofilament yarns. In still further aspects, the first plurality of weft yarns can comprise a combination of yarns that some are multifilament, and some are monofilament yarns. In certain aspects, the second plurality of weft yarns can comprise multifilament yarns. Yet, in other aspects, the second plurality of weft yarns can comprise monofilament yarns. In still further aspects, the second plurality of weft yarns can comprise a combination of yarns that some are multifilament, and some are monofilament yarns.

It is understood that also disclosed herein are aspects where the first plurality of weft yarns is monofilament while the second plurality of weft yarns is multifilament. Yet also disclosed are aspects where the first plurality of weft yarns is multifilament and the second plurality of weft yarns is monofilament. Also disclosed herein are the aspects where the first plurality of weft yarns comprises both monofilament and multifilament yarns, while the second plurality of weft yarns is either monofilament or multifilament. Similarly disclosed are aspects where the second plurality of weft yarns comprises both monofilament and multifilament yarns, while the first plurality of weft yarns is either monofilament or multifilament. Still further disclosed are aspects where the first and the second plurality of weft yarns are the same.

In still further aspects, the first plurality of weft yarns can comprise a number of filaments that are different from the second plurality of weft yarns. It is understood, however, that also disclosed herein are aspects where each weft yarn of the first plurality of weft yarns and/or each yarn of the second plurality of weft yarns can comprise the same number of filaments or a different number of filaments. In still further aspects, the first plurality of weft yarns and/or the second plurality of weft yarns can comprise any number of filaments, for example, it can comprise about 1 to about 200 filaments, including exemplary values of about 2, about 5, about 8, about 10, about 20, about 30, about 40, about 50, about 60, about 70, about 80, about 90, about 100, about 110, about 120, about 130, about 140, about 150, about 160, about 170, about 180, and about 190. It is understood that these values are only exemplary, and the disclosed herein weft yarns can comprise any number of filaments that fall between any two mentioned above values. For example, and without limitations, the yarns in the first plurality of weft yarns and/or the second plurality of weft yarns can also comprise about 7 filaments, about 55 filaments, about 115 filaments, and the like. In other words, the yarns in the first plurality of weft yarns and/or second plurality of weft yarns comprise about 1 to 15 filaments, about 10 to about 100 filaments, or about 20 filaments to about 200 filaments, and the like.

In still further aspects, the first plurality of weft yarns can comprise one or more filaments having the same or different deniers. Yet in still further aspects, each yarn in the first plurality of weft yarns can have filaments having the same denier. While in other aspects, each yarn in the first plurality of weft yarns can have filaments having a different denier. Yet still, in further aspects, the first plurality of weft yarns can comprise yarns that have filaments with the same and/or different deniers.

In still further aspects, the second plurality of weft yarns can comprise one or more filaments having the same or different deniers. Yet in still further aspects, each yarn in the second plurality of weft yarns can have filaments having the same denier. While in other aspects, each yarn in the second plurality of weft yarns can have filaments having a different denier. Yet still, in further aspects, the second plurality of weft yarns can comprise yarns that have filaments with the same and/or different deniers.

In still further aspects, at least a portion of the first plurality of weft yarns has the same or different denier as at least a portion of the second plurality of weft yarns.

In still further aspects, at least one yarn of the first and/or second plurality of weft yarns has a denier of about 10 D to about 50 D, including exemplary values of about 15 D, about 20 D, about 25 D, about 30 D, about 35 D, about 40 D, and about 45 D. It is understood that the at least one yarn of the first and/or second plurality of weft yarns can have any denier that falls between any two foregoing values or within any range formed by any two foregoing values. For example, the at least one yarn of the first and/or second plurality of weft yarns can have denier of about 10D to about 45 D, about 10D to about 40 D, about 10D to about 35 D, about 10D to about 30 D, about 10D to about 25 D, about 10D to about 20 D, about 20 D to about 50 D, about 25 D to about 50 D, about 30 D to about 50 D, about 35 D to about 50 D, about 40 D to about 50 D, about 45 D to about 50 D, and so on.

In still further aspects, the filaments present in the first and/or second plurality of weft yarns can have any diameter suitable for the desired application. In certain aspects, the filament can have a diameter from about 1 μm to about 25 μm, including exemplary values of about 2 μm, about 5 μm, about 7 μm, about 10 μm, about 12 μm, about 15 μm, about 17 μm, about 20 μm, and about 22 μm. It is understood that the filament can have any diameter between any two foregoing values. For example, and without limitation, the filament can have a diameter from about 3 μm to about 8 μm, or from about 11μm to about 22 μm, or from about 15 μm to about 25 μm. Again, it is understood that in order to keep an overall profile of the base layer, disclosed are aspects where the plurality of weft yarns have a relatively low thickness and a diameter in the lower range of the disclosed diameters. It is understood that the thinner profile of the base cloth can allow maximization of the cut pile yarn to ensure the desired sealing properties.

In still further aspects, at least one of the first and/or second plurality of weft yarns comprise a twisted yarn, a plied yarn, a textured yarn, flat, covered yarn, or any combination thereof.

It is understood that if the at least one of the first and/or second plurality of weft yarns comprises the textured yarn, such a textured yarn can be prepared by any known in the art methods. In certain aspects, the textured yarn can be selected from a friction-textured yarn, a pin-textured yarn, an air-textured yarn, a belt-textured yarn, a stuffer box-textured yarn, or any combination thereof. Still, in further exemplary and unlimiting aspects, the textured yarn can be produced by friction texturing, or pin or false twist texturing, air texturing, stuffer box texturing, or any combination thereof. In still further exemplary aspects, the textured yarn is a friction-textured yarn. It is further understood that various textured yarns can have various degrees of texturizing. The degree of texturing is evaluated by measuring crimp contraction, stretch potential, and bulk ratio of the textured yarn. Similarly disclosed herein, weft yarns may comprise no textured yarns.

In certain aspects, at least one yarn in the first and/or second plurality of weft yarns comprises an elastomeric material. In such exemplary and unlimiting aspects, the elastomeric material can comprise one or more thermoplastic polyurethanes, elastomeric polyolefins, silicon-based materials, copolymers thereof, or any combination thereof.

While in still further aspects, at least one yarn in the first and/or second plurality of weft yarns comprises at least one low-melt polymer. For example, and without limitations, the at least one low-melt polymer can comprise one or more of low-melt polyester (or a copolymer thereof), polypropylene, thermoplastic polyurethane, polyamide (Nylon 6, Nylon 6,6, Nylon 12, etc.), and the like, and any combination thereof.

In certain aspects, yarns in the first and/or second plurality of weft yarns can also comprise a composite filament. It is understood that, as used herein, the composite filament relates to a filament that can comprise one or more different materials. In certain aspects, the composite filament is a bi-component filament that can have any configuration, for example, and without limitation, a side-by-side configuration, a core/sheath configuration, a segmented configuration, an islands-in-the-sea configuration, or any combination thereof.

Yet, in still further aspects, the one or more bi-component filaments can comprise the core/sheath configuration. In such exemplary and unlimiting aspects, a sheath can comprise a polymer with a lower melting temperature than a polymer in a core.

It is further understood that the aspects described herein comprise yarns that are biocompatible. Also disclosed herein are aspects that comprise yarns comprising permanent implant-grade polymers. In still further aspects, at least one yarn of the first and/or second plurality of weft yarns can comprise one or more filaments comprising one or more non-resorbable materials. In such exemplary and unlimiting aspects, wherein the one or more non-resorbable materials comprise polyester, co-polyesters, polyamide, polyolefin, polyurethanes, natural fibers, fluoropolymers, polyethers, polyureas, copolymers thereof, metal wires, or any combination thereof.

In still further aspects, the at least one yarn of the first and/or second plurality of weft yarns comprise PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, Co-Cr, NiT, or a combination thereof.

In still further aspects, at least one yarn of the first and/or second plurality of weft yarns comprise one or more filaments comprising one or more bio-resorbable materials. In such exemplary and unlimiting aspects, the one or more bio-resorbable materials comprise PEO, PGA, PLLA, or a combination thereof.

In still further aspects, at least one yarn of the first and/or second plurality of weft yarn is a twisted yarn with about 3 to about 16 turns per inch of yarn, including exemplary values of about 4, about 5, about 6, about 7, about 8, about 9, about 10, about 11, about 12, about 13, about 14, and about 15 turns per inch of yarn. It is understood that the at least one yarn of the first and/or second plurality of weft yarn can have a twist having any value between any two foregoing values or can fall within any range formed by any two foregoing values. For example, and without limitations, the at least one yarn of the first and/or second plurality of weft yarn is a twisted yarn with about 3 to about 15 turns per inch of yarn, about 3 to about 12 turns per inch of yarn, about 3 to about 10 turns per inch of yarn, about 3 to about 8 turns per inch of yarn, about 3 to about 5 turns per inch of yarn, about 5 to about 16 turns per inch of yarn, about 8 to about 16 turns per inch of yarn, about 10 to about 16 turns per inch of yarn, about 12 to about 16 turns per inch of yarn, about 15 to about 16 turns per inch of yarn, and so on.

### PILE YARN

In still further aspects, the disclosed herein textiles comprise a plurality of pile yarn. When the textile is the double cloth, for example, 100 (FIG. 1B), the plurality of pile yarns 106 extends between the top and bottom portions of the cloth such that the plurality of pile yarns 106 extend between the second surface 102b of the first woven base layer 102 and the second surface 104b of the second woven base layer 104; wherein a first portion 106a of the plurality of pile yarns is interwoven with the first woven base layer and a second portion 106b of the plurality of pile yarns is interwoven with the second woven base layer.

In yet still further aspects, when the double cloth is cut, as shown, for example, in FIG. 1C, a separate cloth, as shown in FIG. 2, is formed. In this exemplary cloth, each of the woven base layers comprises a plurality of warp yarns 202, a plurality of weft yarns 204, and a cut pile yarn layer that comprises portions 206 of the plurality of pile yarn.

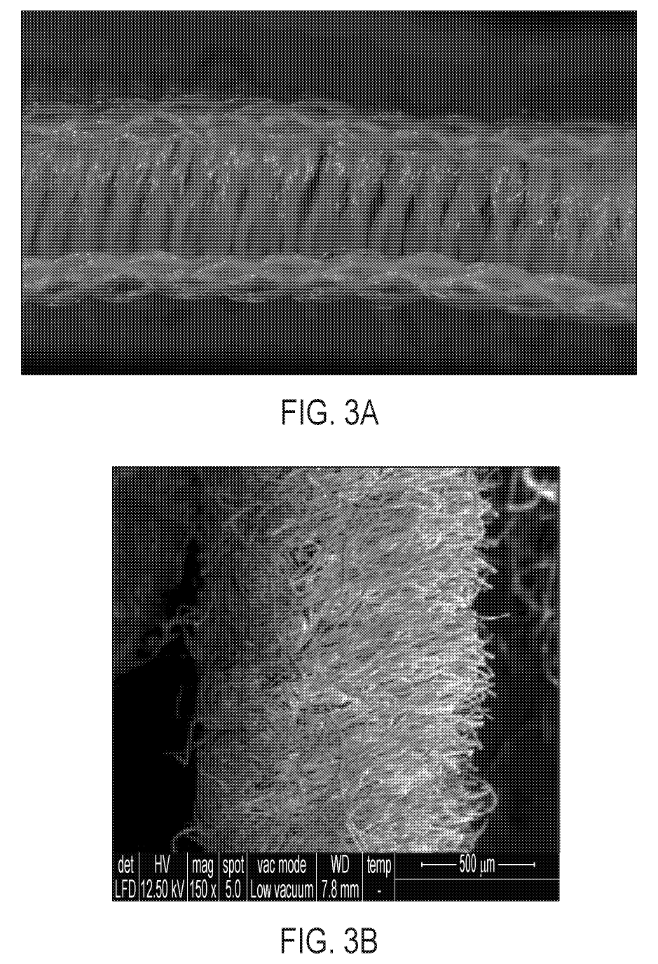

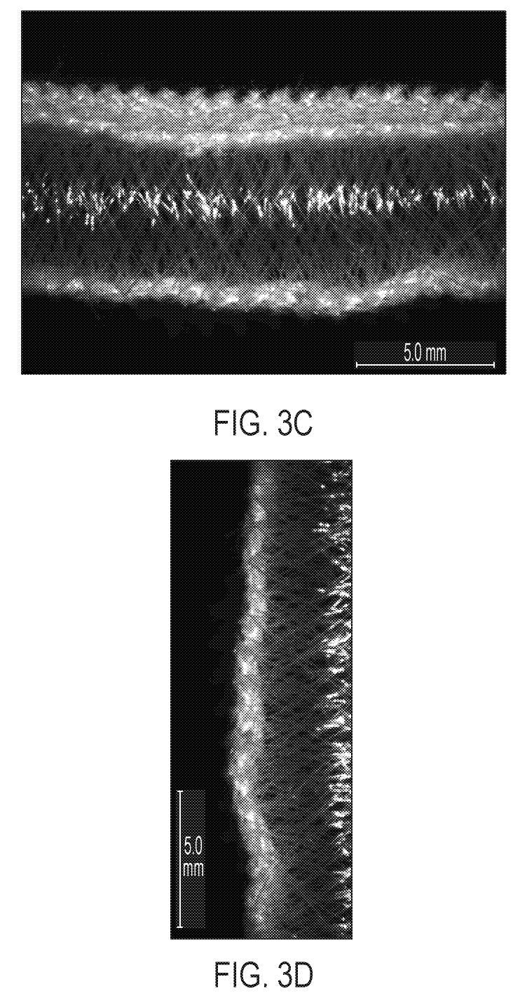

In still further aspects, at least one yarn of the plurality of pile yarns is a multifilament yarn. While in other aspects, at least one yarn of the plurality of pile yarns is a monofilament yarn. In still further aspects, the plurality of pile yarns can comprise a combination of yarns that some are multifilament, and some are monofilament yarns. It is understood that, depending on the type of pile yarn, the covering can have different applications. For example, and without limitations, when the pile yarn is multifilament, the formed covering can be a cushioning covering. Such cushioning coverings can be configured to substantially reduce a PVL and/or increase tissue ingrowth. While in still further aspects, when the pile yarn is a monofilament yarn, such a covering is a frictional element and is configured to increase the retention of the valve in native anatomy or to couple various elements of the valve. For example, in some implementations, the frictional element can be used to anchor into one or more leaflets present in the valve instead of using conventional metal clasps. Some exemplary double cloth materials and separate cloth materials are shown in FIGS. 3A-3D. For example, FIGS. 3A and 3C show photographs of the double cloth materials, wherein the pile yarn is multifilament (FIG. 3A) or monofilament (FIG. 3C). FIGS. 3B (multifilament yarns) and 3D (monofilament yarn) show the pile cut layer after the pile yarn was cut to form separate clothes.

In still further aspects, the plurality of pile yarns can comprise any number of filaments. In still further aspects, the plurality of pile yarns can comprise about 1 to about 200 filaments, including exemplary values of about 2, about 5, about 8, about 10, about 20, about 30, about 40, about 50, about 60, about 70, about 80, about 90, about 100, about 110, about 120, about 130, about 140, about 150, about 160, about 170, about 180, and about 190. It is understood that these values are only exemplary, and the disclosed herein pile yarns can comprise any number of filaments that fall between any two mentioned above values. For example, and without limitations, the yarns in the plurality of pile yarns can also comprise about 7 filaments, about 55 filaments, about 115 filaments, and the like. In other words, the yarns in the plurality of pile can comprise about 1 to 15 filaments, about 10 to about 100 filaments, or about 20 filaments to about 200 filaments, and the like.

In still further aspects, the plurality of pile yarns can comprise one or more filaments having the same or different deniers. Yet, in still further aspects, each yarn in the plurality of pile yarns can have filaments having the same denier. While in other aspects, each yarn in the plurality of pile yarns can have filaments having a different denier. Yet still, in further aspects, the plurality of pile yarns can comprise yarns that have filaments with the same and/or different deniers.

In still further aspects, at least one yarn of the plurality of pile yarns has a denier of about 20 D to about 200 D, including exemplary values of about 25 D, about 30 D, about 35 D, about 40 D, about 45 D, about 50 D, about 55 D, about 60 D, about 65 D, about 70 D, about 75 D, about 80 D, about 85 D, about 90 D, about 95 D, about 100 D, about 105 D, about 110 D, about 115 D, about 120 D, about 125 D, about 130 D, about 135 D, about 140 D, about 145 D, about 150 D, about 155 D, about 160 D, about 165 D, about 170 D, about 175 D, about 180 D, about 185 D, about 190 D, and about 195 D. It is understood that the at least one yarn of the plurality of pile yarns can have any denier that falls between any two foregoing values or within any range formed by any two foregoing values. For example, the at least one yarn of the plurality of pile yarns can have a denier of about 30 D to about 200 D, about 50 D to about 200 D, about 80 D to about 200 D, about 100 D to about 200 D, about 120 D to about 200 D, about 150 D to about 200 D, about 170 D to about 200 D, about 20 D to about 180 D, about 20 D to about 150 D, about 20 D to about 120 D, about 20 D to about 100 D, about 20 D to about 80 D, about 20 D to about 50 D, and so on.

In still further aspects, the filaments present in the plurality of pile yarns can have any diameter suitable for the desired application. In certain aspects, the filament can have a diameter from about 1 μm to about 25 μm, including exemplary values of about 2 μm, about 5 μm, about 7 μm, about 10 μm, about 12 μm, about 15 μm, about 17 μm, about 20 μm, and about 22 μm. It is understood that the filament can have any diameter between any two foregoing values. For example, and without limitation, the filament can have a diameter from about 3 μm to about 8 μm, or from about 11μm to about 22 μm, or from about 15 μm to about 25 μm. It is understood that the finer filament sizes can provide a high surface area that can promote better sealing and, as a result, tissue ingrowth for device integration with the anatomy. Yet in still further unlimited aspects, the finer filaments can enable a high filament count in the yarn that further maximizes the surface area of the pile surface.

In yet still further aspects, at least one yarn of the plurality of pile yarns is a flat yarn, a twisted yarn, a textured yarn, or a combination thereof.

It is understood that if the at least one of the plurality of pile yarns comprises the textured yarn, such a textured yarn can be prepared by any known in the art methods. In certain aspects, the textured yarn can be selected from a friction-textured yarn, a pin-textured yarn, an air-textured yarn, a belt-textured yarn, a stuffer box-textured yarn, or any combination thereof. Still, in further exemplary and unlimiting aspects, the textured yarn can be produced by friction texturing, or pin or false twist texturing, air texturing, stuffer box texturing, or any combination thereof. In still further exemplary aspects, the textured yarn is a friction-textured yarn. It is further understood that various textured yarns can have various degrees of texturizing. The degree of texturing is evaluated by measuring crimp contraction, stretch potential, and bulk ratio of the textured yarn. It is understood that the textured yarn exhibits a crimped or wavy nature of the constituent filaments. Due to this, these yarns provide high bulk and, thus, a higher surface area compared to flat or twisted yarns. Further, based on different texturizing techniques, the level of bulk of yarn differs.

In still further aspects, at least one yarn of the plurality of pile yarns is a twisted yarn with about 2 to about 6 turns per inch of yarn, including exemplary values of about 3, about 4, and about 5 turns per inch of yarn.

It is further understood that the aspects described herein comprise yarns that are biocompatible. Also disclosed herein are aspects that comprise yarns comprising permanent implant-grade polymers. In still further aspects, at least one yarn of the plurality of pile yarns can comprise one or more filaments comprising one or more non-resorbable materials. In such exemplary and unlimiting aspects, wherein the one or more non-resorbable materials comprise polyester, co-polyesters, polyamide, polyolefin, polyurethanes, natural fibers, fluoropolymers, polyethers, polyureas, copolymers thereof, metal wires, or any combination thereof. In some aspects, the metal wires can be flat, smooth, or have a barbed surface.

In still further aspects, the at least one yarn of the plurality of pile yarns comprise PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, cobalt-chromium ( Co-Cr), nitinol (NiT), stainless steel, or a combination thereof.

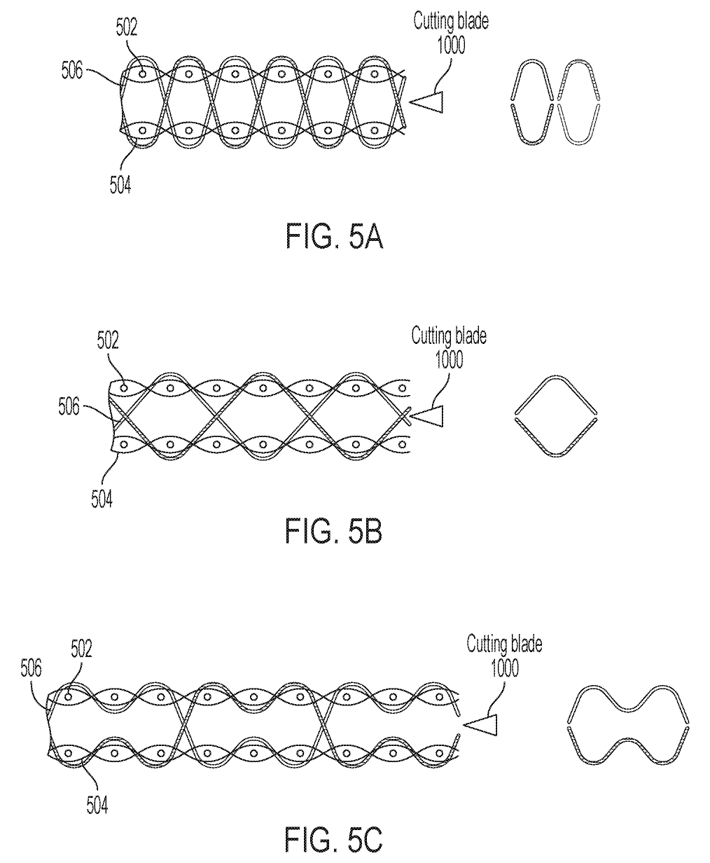

In still further aspects, the pile yarn can be woven such that when the first and/or second cut pile layers are formed, each of the layers can have a V-pattern, U-pattern, W-pattern, or a combination thereof. Exemplary patterns are shown in FIGS. 5A–5C. For example, in FIG. 5A pile yarn 506 is interwoven with the warp yarns 502, and weft yarns 504 to form a V-pattern. In FIG. 5B pile yarn 506 is interwoven with the warp yarns 502 and weft yarns 504 to form a U-pattern. In FIG. 5C pile yarn 506 is interwoven with the warp yarns 502, and weft yarns 504 to form a W-pattern.

In still further aspects, the first cloth and/or second cloth that is formed after cutting the double woven cloth can have about 40 to about 400 ends per inch (EPI), including exemplary values of about 50, about 60, about 70, about 80, about 90, about 100, about 125, about 150, about 175, about 200, about 225, about 250, about 275, about 300, about 325, about 350, and about 375 ends per inch (EPI). It is understood that the first cloth and/or second cloth can have an EPI value that falls between any two foregoing values or within a range formed by any two foregoing values. For example, and without limitations, that EPI can be about 50 to about 400, about 80 to about 400, about 100 to about 400, about 150 to about 400, about 200 to about 400, about 250 to about 400, about 300 to about 400, about 40 to about 350, about 40 to about 300, about 40 to about 250, about 40 to about 200, about 40 to about 150, about 40 to about 100 and so on.

Yet in still further aspects, the first cloth and/or second cloth has about 40 to about 200 picks per inch (PPI), including exemplary values of about 50, about 60, about 70, about 80, about 90, about 100, about 110, about 120, about 130, about 140, about 140, about 160, about 170, about 180, and about 190 picks per inch. It is understood that the first cloth and/or second cloth can have a PPI value that falls between any two foregoing values or within a range formed by any two foregoing values. For example, and without limitations, that EPI can be about 50 to about 200, about 80 to about 200, about 100 to about 200, about 150 to about 200, about 180 to about 200, about 50 to about 200, about 80 to about 200, about 100 to about 200, about 150 to about 200, and so on.

In still further aspects, the first and/or second cloth can have a varying density. It is understood that the density of the cloth can be controlled by various methods known in the art. For example, the density can be controlled by varying the ratio of the number of pile yarns to the number of warp yarns in the first and/or second woven base layers. Yet, in other aspects, the density of the cloth can be controlled by using different sizes of yarn in the top layer and bottom layer during the weaving process. For example, while the number of warp yarns in the top layer and bottom layer can be the same however, the size of the yarn and the number of filaments in the weft yarn can be different.

In still further aspects, and as disclosed above, at least a portion of the first portion of the plurality of pile yarns and at least a portion of the second portion of the plurality of pile yarns are integrally fused with at least a portion of the first woven base layer and/or with at least a portion of the second woven base layer, respectively. It is understood that such a feature assists in securing the pile yarns within the woven base layer and improving the durability of the covering during its positioning in the natural anatomy.

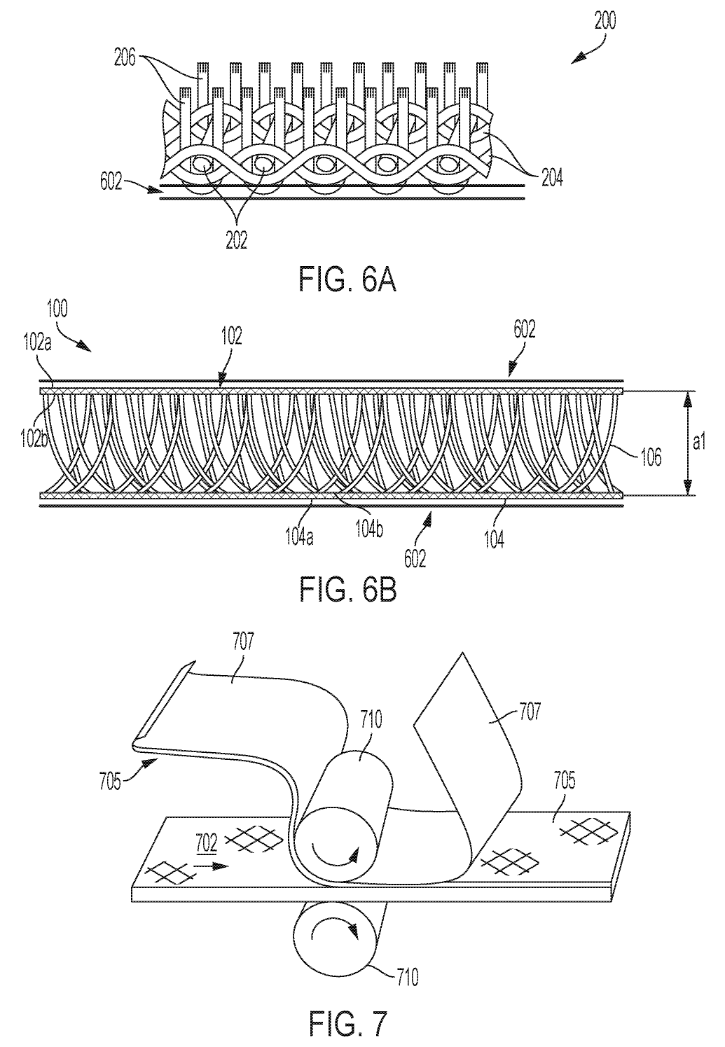

The integral fusing of the pile yarn portions with the woven base layers can be done by any method known in the art. For example, and without limitations, the methods can comprise disposing a layer of an elastomeric material or a low melt polymer on the first surface of the first woven base layer. Various examples of such aspects are shown in FIGS. 6A and 6B. FIG. 6A shows an exemplary as-cut cloth, while FIG. 6B shows an exemplary double cloth before it is cut. It can be seen that a layer of an elastomeric material or a low melt polymer 602 can be disposed on the first surface 102a (or 104a) of the woven base layer. Again, any of the low-melt polymers can be utilized. In certain aspects, for example, and without limitations, the low melt polymers can comprise one or more of low melt polyester (or a copolymer thereof), polypropylene, thermoplastic polyurethane, polyamide (Nylon 6, Nylon 6,6, Nylon 12, etc.), and the like and any combination thereof.

It is understood that if at least some of the first and/or second plurality of warp yarns and/or some of the first and/or second plurality of weft yarns comprise elastomeric or low melt materials, there is no need to dispose another layer of such materials. Just heating the cloth can allow the low-melt material to start melting and integrally infusing the pile yarn portions within the woven base layers. It is understood that the heating can be done in any known in the art methods. For example, the heating rolls can be positioned such that woven double cloth material can pass through these rolls during the weaving process. In such aspects, the heating can be done through contact with the first surfaces of the first and the second woven base layer with the heated rolls. However, it is also understood that heating can be done without the presence of the rolls, for example, by the use of IR or UV heaters. It is also understood that the combination of heating rolls, IR, and UV heaters can also be utilized.

An exemplary and unlimiting method steps of integrally fusing portions of the pile yarn with the woven base layers are shown in FIG. 7. An elastomeric material 705, such as TPU-based coating disposed on a release substrate 707 is disposed on the double woven fabric 702 on a conveyer belt passing through heating rolls 710 to ensure that is TPU-based material is fully melted, and the pile yarns are fused with the woven base layer. In such exemplary and unlimiting aspects, the TPU-based material is present in film form and is attached to a release liner for application purposes. During the lamination process using pressure and heat from the roller, TPU film is softened to melt for a laminate layer by its fusion with the base layer while the release liner is removed.

Similarly, the heating can be done after the full woven cloth is formed and taken from the woven machines, or it can be done during the cutting step or after the double cloth is fully cut and each of the first and the second clothes can be heated separately to form the desired level of integral fusion. It is understood that these exemplary steps can be applicable when the low-melt polymers are used during the weaving process, and they are part of the woven cloth as it is taken from the weaving machine.

In such aspects, the method can further comprise heating the layer of the elastomeric material or the low melt polymer to integrally fuse at least a portion of the first portion of the plurality of pile yarns with at least a portion of the first woven base layer and/or at least a portion of the second portion of the plurality of pile yarns with at least a portion of the second woven base layer. In still further aspects, it also disclosed heating the first woven base layer to integrally fuse at least a portion of the first portion of the plurality of pile yarns with at least a portion of the first woven base layer and/or heating the second woven base layer to integrally fuse at least a portion of the second portion of the plurality of pile yarns with at least a portion of the second woven base layer.

In still further aspects, the formed coverings can be positioned on an outer surface of the frame to form an outer skirt. While in other aspects, the covering can form a covering of an outside valve component. While in still further aspects, the outside valve component comprises one or more anchors on the frame, as described in detail below. In still further aspects, the covering is positioned on an outer surface of the frame to form an outer skirt. Yet, in still further aspects, the covering forms a covering of an outside valve component. While in still further aspects, the outside valve component comprises one or more anchors on the frame.

Also disclosed herein are additional methods of making an implantable prosthetic valve. In these additional, exemplary and unlimiting methods the covering is formed by weaving a double cloth comprising a top portion and a bottom portion, wherein the top portion and bottom portion are spaced at a first length; wherein the top portion comprises a first woven base layer having a first surface and an opposing second surface and comprising a first plurality of warp yarns and a first plurality of weft yarns; wherein the bottom portion comprises a second woven base layer having a first surface and an opposing second surface comprising a second plurality of warp yarns and a second plurality of weft yarns; wherein the first and second woven base layers have a first predetermined pattern; wherein the top portion and bottom portion are coupled with a plurality of pile yarns such that the second surface of the first woven base layer faces the second surface of the second woven base layer, wherein the plurality of pile yarns extend between the second surface of the first woven base layer and the second surface of the second woven base layer along the first length; wherein a first portion of the plurality of pile yarns is interwoven with the first woven base layer and a second portion of the plurality of pile yarns is interwoven with the second woven base layer; wherein the first woven base layer is bio-resorbable; wherein at least a portion of the second portion of the plurality of pile yarns is integrally fused with at least a portion of the second woven base layer.

In this exemplary method, the double cloth material is not cut but positioned on a prosthetic valve frame such that the first surface of the first woven base layer is exposed to the natural anatomy. It is understood that in such aspects, the portion of the plurality of pile yarns that are interwoven with the first woven base layer is not fused with this layer. In still further aspects, the bio-resorbable first woven base layer will resorb in the natural anatomy with time and expose the plurality of pile yarns to the natural anatomy for the desired application.

In such exemplary aspects, the second woven base layer can comprise any of the disclosed above plurality of warp and weft yarns. In yet still further aspects, the plurality of the pile yarn can also comprise any of the disclosed herein pile yarns. In yet other aspects, the first woven base layer comprises only bio-resorbable yarns. Such yarns can be selected from any bio-resorbable materials disclosed above.

In still further aspects, the first length can be any length disclosed above. It is also understood that if needed, an additional layer of an elastomeric material or low melt material can be disposed on the second surface of the second woven base layer and heated to ensure that at least a portion of the pile yarn interwoven with the second woven base layer is integrally fused.

### IMPLANTABLE MEDICAL DEVICE

Paravalvular leak (PVL) is a complication associated with the implantation of a prosthetic heart valve. PVL refers to blood flowing through a channel between the structure of the implanted valve and cardiac tissue as a result of a lack of appropriate sealing. The majority of PVL are crescent, oval, or roundish-shaped, and their track can be parallel, perpendicular, or serpiginous. Transcatheter Heart Valve (THV) procedures generally use either a substantially inelastic woven cloth or a stretchable knitted cloth for PVL sealing.

With the next generation of THV frame designs that have changing frame dimensions, one of the requirements is to have the PVL seal cloth and/or the frame inner cloth to adapt to the changing frame dimensions. The methods disclosed herein allow to provide the coverings that can be used in different frames of implantable devices for different applications.

In certain aspects, disclosed herein is a prosthetic valve comprising: at least one covering comprising: a woven base layer having a first surface and a second surface, and a first predetermined pattern; a cut pile layer, wherein the cut pile layer has a predetermined thickness and a second predetermined pattern; and wherein the cut pile layer comprises a plurality of yarns outwardly extending from the second surface of the woven base layer, wherein at least one yarn of the plurality of yarns present in the cut pile layer is a multifilament yarn or a monofilament yarn, and wherein at least a portion of the plurality of pile yarns is integrally fused with the woven base layer; and wherein the at least one covering is positioned on a valve frame, such that the first surface of the woven base layer is adjacent to the frame.

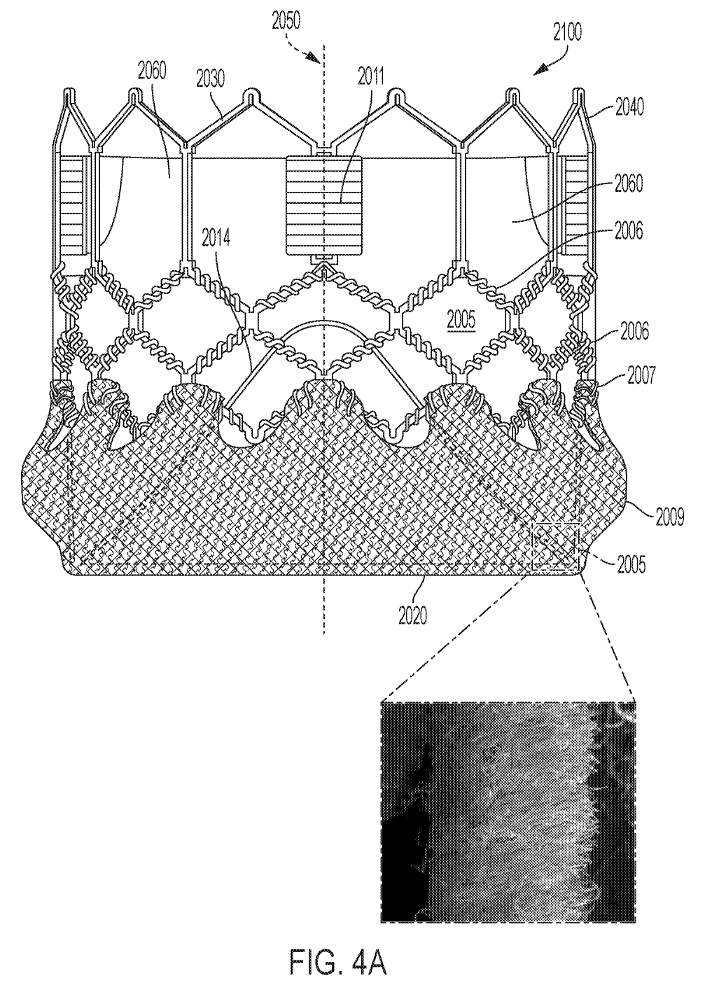

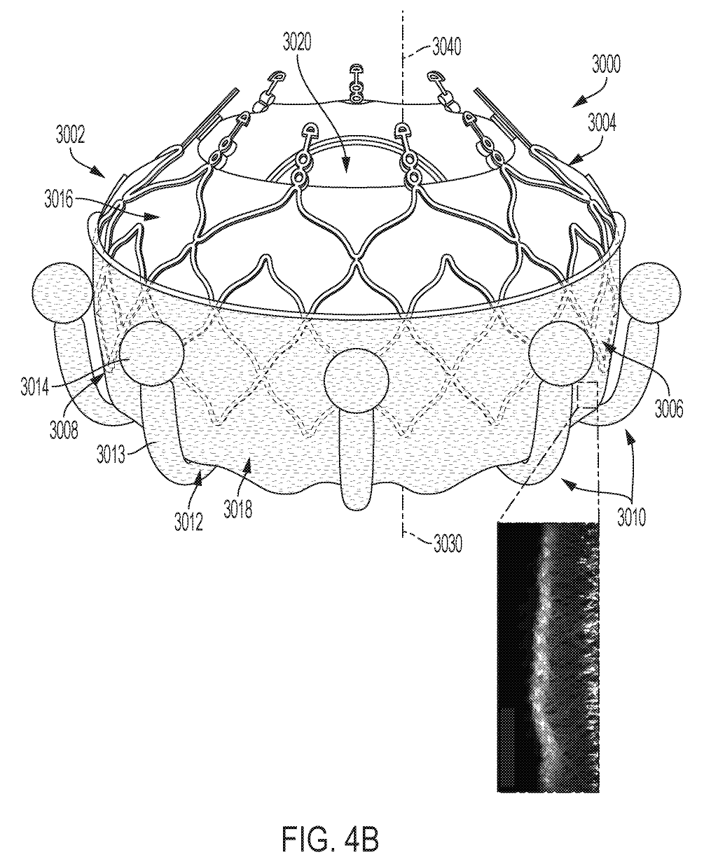

Some exemplary prosthetic devices are shown in FIGS. 4A and 4B. In one aspect and as shown in FIG. 4A disclosed herein is an implantable prosthetic valve 2100, comprising: an annular frame 2040, wherein the frame has an inflow end 2020 and an outflow end 2030, and a central longitudinal axis 2050 extending from the inflow end to the outflow end. An exemplary prosthetic heart valve 2100 is also described in U.S. Patent No. 10,463,484, titled “Prosthetic Heart Valve Having Leaflet Inflow Below Frame,” which is incorporated herein by reference. The illustrated prosthetic valve 2100 is adapted to be implanted in the native aortic annulus, although in other aspects, it can be adapted to be implanted in the other native annuluses of the heart (the mitral valve, pulmonary valve, and tricuspid valve).

In still further aspects, the implantable prosthetic valve also comprises an inner skirt 2005 having an inner surface and an outer surface and is positioned along the inner surface of the frame. In still further aspects, the valve comprises a leaflet structure 2060 comprising one or more leaflets, having an inner surface and an outer surface, and positioned at the inner surface of the annular frame. In still further aspects, the valve can optionally comprise an outer skirt. The outer skirt 2009 as shown, for example, in FIG. 4A has an inner surface and an outer surface and is positioned at the outer surface of the annular frame 2040. The outer skirt can also be attached to the frame with one or more sutures 2007. The valve can also comprise additional sutures, for example, 2006 and 2014, that run along various portions of the device. In still further aspects, the sutures can be used to attach at least a portion of the inner skirt and/or at least a portion of the leaflet structure and/or at least a portion of the outer skirt, if present, to at least a frame of an implantable prosthetic valve. In yet still further aspects, the sutures can be used to attach various components of the valve together.

In aspects disclosed herein, the covering formed by the methods described above can be used as an outer skirt 2009. For example, in this specific aspect, the outer skirt can comprise a separate cloth having the first and/or second woven base layer and the first and/or second cut pile layer, respectively. In such aspects, the first and/or second woven base layer can comprise any of the warp or weft yarns disclosed above. In yet further aspects, the cut pile yarn layer can comprise portions of the plurality of pile yarns comprising multifilament yarns. In such exemplary and unlimiting aspects, the multifilament yarn present in the cut pile yarn layer can comprise any of the disclosed above materials, number of filaments, size, or denier of the filaments. In yet still further aspects, the thickness of this cut pile yarn can have any of the disclosed above values.

In still further aspects, the outer skirt 2009 can comprise a double cloth as described above, where the first woven base layer is made of bio-resorbable material, and the second woven base layer and the plurality of pile yarns comprise any of the disclosed above materials. It is understood that in this exemplary aspect, the bio-resorbable first woven base layer is expected to resorb in the native anatomy with time, exposing the plurality of pile yarns to the native anatomy.

In certain aspects, the prosthetic valve 2100 can comprise a leaflet structure 2060. In certain aspects, the leaflet structure can comprise one or more leaflets, each of which can be arranged to collapse in a tricuspid arrangement. The certain aspects, an edge of the leaflet structure 2060 can have an undulating, curved scalloped shape. Moreover, by virtue of the scalloped shape, folds, and ripples at the belly of each leaflet, which can cause early calcification in those areas, can be eliminated or at least minimized. Leaflets can have various other shapes and/or configurations in other aspects. It is understood, however, that the leaflets of the leaflet structure need not have a V-shaped or scalloped inflow edge, and instead, each leaflet can have a square or rectangular shape defining a straight inflow edge.

As shown in FIG. 4A, the leaflets 2060 can be secured with the one or more sutures 2014 to the frame 2040. The frame 2040 can be made of any of various suitable plastically-expandable materials (e.g., stainless steel, cobalt chromium, etc.) or self-expanding materials (e.g., nitinol) as is known in the art. When constructed of a plastically-expandable material, the frame 2040 can be crimped to a radially compressed state on a delivery catheter and then expanded inside a patient by an inflatable balloon or equivalent expansion mechanism. When constructed of a self-expandable material, the frame 2040 can be crimped to a radially compressed state and restrained in the compressed state by insertion into a sheath or equivalent mechanism of a delivery catheter. Once inside the body, the valve can be advanced from the delivery sheath, which allows the valve to expand to its functional size. Suitable plastically-expandable materials that can be used to form the frame 2040 include, without limitation, stainless steel, a nickel-based alloy (e.g., a cobalt-chromium or a nickel-cobalt-chromium alloy), polymers, or combinations thereof. In particular aspects, frame 2040 can be made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum by weight. In aspects when MP35N is used as the frame material, less material is needed to achieve the same or better performance in radial and crush force resistance, fatigue resistance, and corrosion resistance. Moreover, since less material is required, the crimped profile of the frame can be reduced, thereby providing a lower profile valve assembly for percutaneous delivery to the treatment location in the body.

The frame 2040 can have other configurations or shapes in other aspects.

The leaflets can be sutured together to form the leaflet structure 2060, which can then be secured to the frame 2040. It is understood that when the leaflets are sutured together, each of the leaflets can comprise any of the disclosed herein sutures if desired. However, also disclosed herein are the aspects, when different from the disclosed one or more sutures are used to secure the leaflet structure within the valve. In still further exemplary and unlimiting aspects, the leaflets of the leaflet structure 2060 can be secured to one another at their adjacent sides to form commissures 2011 of the leaflet structure. It is understood that sutures disclosed herein can also be used to form such commissures. Yet, in other aspects, sutures different from those disclosed herein sutures can also be used.

In still further exemplary aspects, the one or more leaflets of the leaflet structure 2060 can be attached to the inner skirt 2005, with sutures 2014. The suture 2014 can track the curvature of the bottom edge of the leaflet structure 2060 and are collectively referred to as the scallop line.

The inner skirt 2005 can have a plurality of functions, which can include assisting in securing the leaflet structure 2060 and/or the outer skirt 2009 to the frame 2040 and to assist in forming a good seal between the valve 2100 and the native annulus by blocking the flow of blood below the lower edges of the leaflets. It is understood that any known in the art configuration/construction of the skirt can be used. For example, and without limitation, the configuration/construction of the skirt can comprise a textile that is braided, knitted, woven and/or nonwoven. The inner skirt 2005 can comprise a tough, tear-resistant material such as polyethylene terephthalate (PET), although various other synthetic or natural materials can be used. The thickness of the skirt is desirably less than 6 mils or 0.15 mm, and desirably less than 4 mils or 0.10 mm, and even more desirably about 2 mils or 0.05 mm, and even still more desirably about 1.1 mils or 0.03 mm. In particular aspects, the skirt 2005 can have a variable thickness, for example, the skirt can be thicker at its edges than at its center. In one implementation, the skirt 2005 can comprise a PET skirt having a thickness of about 0.07 mm at its edges and about 0.06 mm at its center. The thinner skirt can provide for better crimping performances while still providing good perivalvular sealing. In still further aspects, the inner skirt 2005 can have any of the disclosed coatings herein. In certain aspects, only an inner surface of the inner skirt 2005 can have the disclosed herein coating. While in other aspects, both surfaces of the inner skirt can have the same coating.

The outer skirt, as shown in FIG. 4A can serve as a cushioning covering and can also behave as a tissue ingrowth graft if desired.

FIG. 4B shows a different exemplary valve that utilizes coverings produced by the methods disclosed herein. FIG. 4B is configured to be deployed to a native valve of the heart.

Referring to FIG. 4B, in this aspect, the prosthetic valve 3000, comprises a frame 3002, having an upper region 3004, an intermediate region 3006, and a lower region 3008. The valve further comprises a plurality of prosthetic leaflets 3020, an inner skirt 3016, and an outer skirt 3018. In still further aspects, the implantable medical device has an inflow end 3040 and an outflow end 3030, and the one or more anchors 3010 are positioned at the outflow end of the prosthetic valve. The anchors 3010 shown in the FIG. 4B are positioned within the disclosed herein woven articles. The anchors can be defined by a neck 3012, an arm 3013, and a tip 3014 (or head).

In still further aspects, the implantable medical device is configured to be deployed to a native valve of a heart, wherein the prosthetic valve further comprises the plurality of prosthetic valve leaflets as shown herein, wherein the one or more anchors are coupled to the plurality of prosthetic valve leaflets, and each is configured to anchor to a portion of the heart.

As disclosed in detail herein, the implantable medical device can further comprise an outer skirt 3018 (FIG. 4B). The outer skirt can also be used for a paravalvular leak seal or as a friction element. The disclosed herein coverings can be positioned as the outer skirt 3018 or as coverings of the anchors. In such aspects, the disclosed coverings can behave as frictional elements assisting the incorporation of the valve within the natural anatomy. In such aspects, the plurality of pile yarns used for these coverings are monofilament yarns. All other components of the coverings can comprise any of the disclosed elements. This fictional covering can have any of the disclosed above configurations and materials as long as the pile yarn is a monofilament yarn.

### EXEMPLARY ASPECTS

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1. A method of forming an implantable prosthetic valve, wherein the method comprises: (a) forming a valve covering material, wherein the step of forming comprises: (i) weaving a double cloth comprising a top portion and a bottom portion, wherein the top portion and bottom portion are spaced at a first length; wherein the top portion comprises a first woven base layer having a first surface and an opposing second surface and comprising a first plurality of warp yarns and a first plurality of weft yarns; wherein the bottom portion comprises a second woven base layer having a first surface and an opposing second surface comprising a second plurality of warp yarns and a second plurality of weft yarns; wherein the top portion and bottom portion are coupled with a plurality of pile yarns such that the second surface of the first woven base layer faces the second surface of the second woven base layer, wherein the plurality of pile yarns extend between the second surface of the first woven base layer and the second surface of the second woven base layer along the first length; wherein a first portion of the plurality of pile yarns is interwoven with the first woven base layer, and a second portion of the plurality of pile yarns is interwoven with the second woven base layer; (ai) cutting through the plurality of pile yarns transversely to the first length to form: a first cloth comprising the first woven base layer having a first predetermined pattern and a first cut pile layer comprising a portion of the plurality of pile yarns and extending outwardly from the second surface of the first woven base layer, wherein the first cut pile layer has a second predetermined pattern and a first predetermined thickness; a second cloth material comprising the second woven base layer having the first predetermined pattern and a second cut pile layer comprising a portion of the plurality of pile yarns and extending outwardly from the second surface of the second woven base layer, wherein the second cut pile layer has the second predetermined pattern and a second predetermined thickness; and wherein at least a portion of the first portion of the plurality of pile yarns is integrally fused with at least a portion of the first woven base layer; and/or wherein at least a portion of the second portion of the plurality of pile yarns is integrally fused with at least a portion of the second woven base layer; and (b) positioning at least one of the first and/or second cloth materials on a prosthetic valve frame to form the valve covering such that at least a portion of the first and/or second cut pile layer is exposed to a natural anatomy.

Example 2. The method of any examples herein, particularly example 1, wherein the first predetermined thickness is the same as the second predetermined thickness.

Example 3. The method of any examples herein, particularly example 1, wherein the first predetermined thickness is larger than the second predetermined thickness.

Example 4. The method of any examples herein, particularly example 1, wherein the first predetermined thickness is smaller than the second predetermined thickness.

Example 5. The method of any one of any examples herein, particularly example of any examples herein, particularly examples 1–4, wherein the first predetermined pattern of the first and/or the second woven base layer comprises a plain 1:1 pattern, warp rib 2/1, warp rib 2/2, plain weave, and its derivatives or any combination thereof.

Example 6. The method of any one of any examples herein, particularly example of any examples herein, particularly examples 1–5, wherein the first plurality of warp yarns and the second plurality of warp yarns comprise the same or different material.

Example 7. The method of any one of any examples herein, particularly example of any examples herein, particularly examples 1–6, wherein the first plurality of warp yarns comprise multifilament or monofilament yarns.

Example 8. The method of any one of any examples herein, particularly example of any examples herein, particularly examples 1–7, wherein the second plurality of warp yarns comprise multifilament or monofilament yarns.

Example 9. The method of any one of any examples herein, particularly examples 7 or 8, wherein the first plurality of warp yarns comprises a number of filaments that is the same or different from the second plurality of warp yarns.

Example 10. The method of any one of any examples herein, particularly examples 1–9, wherein the first plurality of warp yarns comprise one or more filaments having the same or different denier.

Example 11. The method of any one of any examples herein, particularly examples 1–10, wherein the second plurality of warp yarns comprise one or more filaments having the same or different denier.

Example 12. The method of any one of any examples herein, particularly examples 1–11, wherein at least a portion of the first plurality of warp yarns has the same or different denier as at least a portion of the second plurality of warp yarns.

Example 13. The method of any one of any examples herein, particularly examples 1–12, wherein the first plurality of weft yarns and the second plurality of weft yarn comprise the same or different material.

Example 14. The method of any one of any examples herein, particularly examples 1–13, wherein the first plurality and second plurality of weft yarn comprises a multifilament yarn.

Example 15. The method of any one of any examples herein, particularly examples 1–13, wherein the first plurality and second plurality of weft yarn comprises a monofilament yarn.

Example 16. The method of any one of any examples herein, particularly examples 1–15, wherein the second predetermined pattern of the first and/or the second cut pile layers comprises a V-pattern, U-pattern, W-pattern, or a combination thereof.

Example 17. The method of any one of any examples herein, particularly examples 1–16, wherein at least one yarn of the plurality of pile yarns is a multifilament yarn.

Example 18. The method of any examples herein, particularly example 17, wherein the covering is a cushioning covering.

Example 19. The method of any examples herein, particularly example 17 or 18, wherein the covering is configured to substantially reduce a PVL and/or increase tissue ingrowth.

Example 20. The method of any one of any examples herein, particularly examples 1–19, wherein at least one yarn of the plurality of pile yarns is a monofilament yarn.

Example 21. The method of any examples herein, particularly example 20, wherein the covering is a frictional element configured to increase the retention of the valve in native anatomy or to couple various elements of the valve.

Example 22. The method of any one of any examples herein, particularly examples 1–21, wherein the covering is positioned on an outer surface of the frame to form an outer skirt.

Example 23. The method of any one of one of any examples herein, particularly examples 1–22, wherein the covering forms a covering of an outside valve component.

Example 24. The method of any examples herein, particularly example 23, wherein the outside valve component comprises one or more anchors on the frame.

Example 25. The method of any one of any examples herein, particularly examples 1–24, wherein at least one of the first and/or second plurality of warp yarns comprise a twisted yarn, a textured yarn, flat, or any combination thereof.

Example 26. The method of any one of any examples herein, particularly examples 1–25, wherein at least one yarn in the first or second plurality of warp yarns comprises an elastomeric material.

Example 27. The method of any one of any examples herein, particularly examples 1–26, wherein at least one yarn in the first or second plurality of warp yarns comprises at least one low melt polymer.

Example 28. The method of any one of any examples herein, particularly examples 1–27, wherein at least one yarn in the first and/or second plurality of warp yarns comprises one or more bi-component filaments.

Example 29. The method of any examples herein, particularly example 28, wherein the one or more bi-component filaments have a core/sheath configuration, side-by-side configuration, lobal configuration, island-in- the sea configuration, segmented configuration, or any combination thereof.

Example 30. The method of any examples herein, particularly example 29, wherein the one or more bi-component filaments comprise the core/sheath configuration and wherein a sheath comprises a polymer with a lower melting temperature than a polymer in a core.

Example 31. The method of any one of any examples herein, particularly examples 26–30, wherein the elastomeric material comprises one or more thermoplastic polyurethanes, elastomeric polyolefins, silicon-based materials, copolymers thereof, or any combination thereof.

Example 32. The method of any one of any examples herein, particularly examples 1–31, wherein at least one yarn of the first and/or second plurality of warp yarns comprise one or more filaments comprising one or more non-resorbable materials.

Example 33. The method of any examples herein, particularly example 32, wherein the one or more non-resorbable materials comprise polyester, co-polyesters, polyamide, polyolefin, polyurethanes, natural fibers, fluoropolymers, polyethers, polyureas, copolymers thereof, metal wires, or any combination thereof.

Example 34. The method of any examples herein, particularly example 32 or 33, wherein the at least one yarn of the first and/or second plurality of warp yarns comprise a PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, cobalt-chromium (Co-Cr), nitinol (NiT), stainless steel or a combination thereof.

Example 35. The method of any one of any examples herein, particularly examples 1–34, wherein at least one yarn of the first and/or second plurality of warp yarns comprise one or more filaments comprising one or more bio-resorbable materials.

Example 36. The method of any examples herein, particularly example 35, wherein the one or more bio-resorbable materials comprise PEO, PGA, PLLA, or a combination thereof.

Example 37. The method of any one of any examples herein, particularly examples 1–36, wherein at least one yarn of the first and/or second plurality of warp yarn is a twisted yarn with about 3 to about 16 turns per inch of yarn.

Example 38. The method of any one of any examples herein, particularly examples 1–37, wherein at least one yarn of the first and/or second plurality of warp yarns has a denier of about 15 D to about 50 D.

Example 39. The method of any one of any examples herein, particularly examples 1–38, wherein at least one yarn of the first and/or second plurality of warp yarns comprises about 1 filament to about 200 filaments.

Example 40. The method of any one of any examples herein, particularly examples 1–39, wherein at least one of the first and/or second plurality of weft yarns comprise a twisted yarn, a plied yarn, a textured yarn, flat, covered yarn, or any combination thereof.

Example 41. The method of any examples herein, particularly example 40, wherein at least one yarn in the first and/or second plurality of weft yarns comprises an elastomeric material.

Example 42. The method of any one of any examples herein, particularly examples 1–41, wherein at least one yarn in the first or second plurality of weft yarns comprises at least one low melt polymer.

Example 43. The method of any one of any examples herein, particularly examples 1–42, wherein at least one yarn in the first or second plurality of weft yarns comprises one or more of bi-component filament.

Example 44. The method of any examples herein, particularly example 43, wherein the one or more bi-component filaments have a core/sheath configuration, side-by-side configuration, lobal configuration, island-in- the sea configuration, segmented configuration, or any combination thereof.

Example 45. The method of any examples herein, particularly example 44, wherein the one or more bi-component filaments comprise the core/sheath configuration and wherein a sheath comprises a polymer with a lower melting temperature than a polymer in a core.

Example 46. The method of any one of any examples herein, particularly examples 41–45, wherein the elastomeric material comprises one or more thermoplastic polyurethanes, elastomeric polyolefins, silicon-based materials, copolymers thereof, or any combination thereof.

Example 47. The method of any one of any examples herein, particularly examples 1–46, wherein at least one yarn of the first and/or second plurality of weft yarns comprise one or more filaments comprising one or more non-resorbable materials.

Example 48. The method of any examples herein, particularly example 47, wherein the one or more non-resorbable materials comprise polyester, co-polyesters, polyamide, polyolefin, polyurethanes, natural fibers, fluoropolymers, polyethers, polyureas, copolymers thereof, metal wires, or any combination thereof.

Example 49. The method of any examples herein, particularly example 47 or 48, wherein the at least one yarn of the first and/or second plurality of weft yarns comprise PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, cobalt-chromium (Co-Cr), nitinol (NiT), stainless steel, or a combination thereof.

Example 50. The method of any one of any examples herein, particularly examples 1–49, wherein at least one yarn of the first and/or second plurality of weft yarns comprise one or more filaments comprising one or more bio-resorbable materials.

Example 51. The method of any examples herein, particularly example 50, wherein the one or more bio-resorbable materials comprise PEO, PGA, PLLA, or a combination thereof.

Example 52. The method of any one of any examples herein, particularly examples 1–51, wherein at least one yarn of the first and/or second plurality of weft yarn is a twisted yarn with about 3 to about 16 turns per inch of yarn.

Example 53. The method of any one of any examples herein, particularly examples 1–52, wherein at least one yarn of the first and/or second plurality of weft yarns has a denier of about 10 D to about 50 D.

Example 54. The method of any one of any examples herein, particularly examples 1–53, wherein at least one yarn of the first and/or second plurality of weft yarns comprises about 1 filament to about 200 filaments.

Example 55. The method of any one of any examples herein, particularly examples 1–54 wherein at least one yarn of the plurality of pile yarns is a flat yarn, a twisted yarn, a textured yarn, or a combination thereof.

Example 56. The method of any examples herein, particularly example 55, wherein the at least one yarn of the plurality of pile yarns is the twisted yarn having 2 to 6 turns per inch of the yarn.

Example 57. The method of any examples herein, particularly example 55 or 56, wherein the at least one yarn of the plurality of pile yarns comprises polyester, polyamide, polyolefin, fluoropolymer, metal wires, or any combination thereof.

Example 58. The method of any examples herein, particularly example 57, wherein the at least one yarn of the plurality of pile yarns comprises a PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, or a combination thereof.

Example 59. The method of any one of any examples herein, particularly examples 1–58, wherein at least one yarn of the plurality of pile yarns has a denier of about 20 deniers to about 200 deniers.

Example 60. The method of any one of any examples herein, particularly examples 1–59, wherein at least one yarn of the plurality of pile yarns has a filament count of about 1 to about 200.

Example 61. The method of any one of any examples herein, particularly examples 1–60, wherein the first cloth and/or second cloth has about 40 to about 400 ends per inch (EPI).

Example 62. The method of any one of any examples herein, particularly examples 1–61, wherein the first cloth and/or second cloth has about 40 to about 200 picks per inch (PPI).

Example 63. The method of any one of any examples herein, particularly examples 1–62, wherein the first and/or second cloth exhibits a varying density.

Example 64. The method of any one of any examples herein, particularly examples 1–63, wherein the first and/or second predetermined thicknesses of the first and/or second cut pile layer is about 0.1 mm to about 5 mm.

Example 65. The method of any one of any examples herein, particularly examples 1–64, wherein the method comprises disposing a layer of an elastomeric material or a low melt polymer on the first surface of the first woven base layer.

Example 66. The method of any one of any examples herein, particularly examples 1–65, wherein the method comprises disposing a layer of an elastomeric material or a low melt polymer on the first surface of the second woven base layer.

Example 67. The method of any one of any examples herein, particularly examples 65 or 66, heating the layer of the elastomeric material or the low melt polymer to integrally fuse at least a portion of the first portion of the plurality of pile yarns with at least a portion of the first woven base layer and/or at least a portion of the second portion of the plurality of pile yarns with at least a portion of the second woven base layer.

Example 68. The method of any one of any examples herein, particularly examples 26–67, heating the first woven base layer to integrally fuse at least a portion of the first portion of the plurality of pile yarns with at least a portion of the first woven base layer and/or heating the second woven base layer to integrally fuse at least a portion of the second portion of the plurality of pile yarns with at least a portion of the second woven base layer.

Example 69. The method of any one of any examples herein, particularly examples 67 or 68, wherein the heating comprises exposure to an IR heater, a UV heater, heater rollers, or a combination thereof.

Example 70. The method of any one of any examples herein, particularly examples 67–69, wherein the heating is performed during the weaving step.

Example 71. The method of any one of any examples herein, particularly examples 67–70, wherein the heating is performed during the cutting step.

Example 72. The method of any one of any examples herein, particularly examples 67–71, wherein the heating is performed after the cutting step.

Example 73. A method of forming an implantable prosthetic valve, wherein the method comprises: (a) forming a valve covering material, wherein the step of forming comprises: weaving a double cloth comprising a top portion and a bottom portion, wherein the top portion and bottom portion are spaced at a first length; wherein the top portion comprises a first woven base layer having a first surface and an opposing second surface and comprising a first plurality of warp yarns and a first plurality of weft yarns; wherein the bottom portion comprises a second woven base layer having a first surface and an opposing second surface comprising a second plurality of warp yarns and a second plurality of weft yarns; wherein the first and second woven base layers have a first predetermined pattern; wherein the top portion and bottom portion are coupled with a plurality of pile yarns such that the second surface of the first woven base layer faces the second surface of the second woven base layer, wherein the plurality of pile yarns extend between the second surface of the first woven base layer and the second surface of the second woven base layer along the first length; wherein a first portion of the plurality of pile yarns is interwoven with the first woven base layer and a second portion of the plurality of pile yarns are interwoven with the second woven base layer; wherein the first woven base layer is bio-resorbable; wherein at least a portion of the second portion of the plurality of pile yarns is integrally fused with at least a portion of the second woven base layer; and (b) positioning the covering on a prosthetic valve frame to form the valve covering such that the first surface of the first woven base layer is exposed to a natural anatomy.

Example 74. A prosthetic valve comprising: at least one covering comprising: a woven base layer having a first surface and a second surface, and a first predetermined pattern; a cut pile layer, wherein the cut pile layer has a predetermined thickness and a second predetermined pattern; and wherein the cut pile layer comprises a plurality of yarns outwardly extending from the second surface of the woven base layer, wherein at least one yarn of the plurality of yarns present in the cut pile layer is a multifilament yarn or a monofilament yarn, and wherein at least a portion of the plurality of pile yarns is integrally fused with the woven base layer; and wherein the at least one covering is positioned on a valve frame, such that the first surface of the woven base layer is adjacent to the frame.

Example 75. The prosthetic valve of any examples herein, particularly example 74, wherein the first predetermined pattern comprises a plain 1:1 pattern, warp rib 2/1, warp rib 2/2, plain weave, and its derivatives or any combination thereof.

Example 76. The prosthetic valve of any one of any examples herein, particularly examples 74–75, wherein the second predetermined pattern comprises a V-pattern, U-pattern, W-pattern, or a combination thereof.

Example 77. The prosthetic valve of any one of any examples herein, particularly examples 74–76, wherein the at least one yarn of the plurality of yarns present in the cut pile layer is a multifilament yarn.

Example 78. The prosthetic valve of any examples herein, particularly example 77, wherein the covering is a cushioning covering configured to substantially reduce the PVL and/or increase tissue ingrowth.

Example 79. The prosthetic valve of any one of any examples herein, particularly examples 74–78, wherein the covering is positioned on an outer surface of the valve frame to form an outer skirt of the prosthetic valve or positioned on an outside valve component.

Example 80. The prosthetic valve of any one of any examples herein, particularly examples 74–76, wherein the at least one yarn of the plurality of yarns present in the cut pile layer is a monofilament yarn.

Example 81. The prosthetic valve of any examples herein, particularly example 80, wherein the covering is a frictional element configured to increase the retention of the valve in native anatomy or to couple various elements of the valve.

Example 82. The prosthetic valve of any one of any examples herein, particularly examples 80 or 81, wherein the covering is positioned on an outer surface of the valve frame to form an outer skirt of the prosthetic valve or positioned on an outside valve component.

Example 83. The prosthetic valve of any examples herein, particularly example 82, wherein the outside valve component comprises one or more anchors present on the frame.

Example 84. The prosthetic valve of any one of any examples herein, particularly examples 74–83, wherein the woven base layer comprises a plurality of warp yarns and a plurality of weft yarns.

Example 85. The prosthetic valve of any examples herein, particularly example 84, wherein at least one of the plurality of warp yarns comprise a twisted yarn, a textured yarn, flat, or any combination thereof.

Example 86. The prosthetic valve of any one of examples herein, particularly examples 84–85, wherein at least one yarn in the plurality of warp yarns comprises an elastomeric material.

Example 87. The prosthetic valve of any one of examples herein, particularly examples 84–86, wherein at least one yarn in the plurality of warp yarns comprises at least one low melt polymer.

Example 88. The prosthetic valve of any one of examples herein, particularly examples 84–87, wherein at least one yarn in the plurality of warp yarns comprises one or more of bi-component filaments.

Example 89. The prosthetic valve of any one of examples herein, particularly example 88, wherein the one or more of the bi-component filaments have core/sheath configuration, side-by-side configuration, lobal configuration, island-in- the sea configuration, segmented configuration, or any combination thereof.

Example 90. The prosthetic valve of any one of examples herein, particularly example 89, wherein the one or more bi-component filaments comprise the core/sheath configuration and wherein a sheath comprises a polymer with a lower melting temperature than a polymer in a core.

Example 91. The prosthetic valve of any one of examples herein, particularly examples 86–90, wherein the elastomeric material comprises one or more thermoplastic polyurethanes, elastomeric polyolefins, silicon-based materials, copolymers thereof, or any combination thereof.

Example 92. The prosthetic valve of any one of examples herein, particularly examples 85–91, wherein at least one yarn of the plurality of warp yarns comprise one or more filaments comprising one or more non-resorbable materials.

Example 93. The prosthetic valve of any one of examples herein, particularly example 92, wherein the one or more non-resorbable materials comprise polyester, co-polyesters, polyamide, polyolefin, polyurethanes, natural fibers, fluoropolymers, polyethers, polyureas, copolymers thereof, metal wires, or any combination thereof.

Example 94. The prosthetic valve of any one of examples herein, particularly example 92 or 93, wherein the at least one yarn of the plurality of warp yarns comprise a PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, cobalt-chromium (Co-Cr), nitinol (NiT), stainless steel, or a combination thereof.

Example 95. The prosthetic valve of any one of examples herein, particularly examples 84–94, wherein at least one yarn of the plurality of warp yarn is a twisted yarn with about 3 to about 16 turns per inch of yarn.

Example 96. The prosthetic valve of any one of examples herein, particularly examples 84–95, wherein at least one yarn of the plurality of warp yarns has a denier of about 15 D to about 50 D.

Example 97. The prosthetic valve of any one of examples herein, particularly examples 84–96, wherein at least one yarn of the plurality of warp yarns comprises about 1 filament to about 200 filaments.

Example 98. The prosthetic valve of any one of examples herein, particularly examples 84–97, wherein at least one of the plurality of weft yarns comprise a twisted yarn, a plied yarn, a textured yarn, flat, covered yarn, or any combination thereof.

Example 99. The prosthetic valve of any one of examples herein, particularly example 98, wherein at least one yarn in the plurality of weft yarns comprises an elastomeric material.

Example 100. The prosthetic valve of any one of examples herein, particularly examples 84–99, wherein at least one yarn in the plurality of weft yarns comprises at least one low melt polymer.

Example 101. The prosthetic valve of any one of examples herein, particularly examples 84–100, wherein at least one yarn in the plurality of weft yarns comprises one or more of bi-component filaments.

Example 102. The prosthetic valve of any one of examples herein, particularly example 101, wherein the one or more of the bi-component filaments have a core/sheath configuration, side-by-side configuration, lobal configuration, island-in- the sea configuration, segmented configuration, or any combination thereof.

Example 103. The prosthetic valve of any one of examples herein, particularly example 102, wherein the one or more bi-component filaments comprise the core/sheath configuration and wherein a sheath comprises a polymer with a lower melting temperature than a polymer in a core.

Example 104. The prosthetic valve of any one of examples herein, particularly examples 99–103, wherein the elastomeric material comprises one or more thermoplastic polyurethanes, elastomeric polyolefins, silicon-based materials, copolymers thereof, or any combination thereof.

Example 105. The prosthetic valve of any one of examples herein, particularly examples 84–104, wherein at least one yarn of the plurality of weft yarns comprise one or more filaments comprising one or more non-resorbable materials.

Example 106. The prosthetic valve of any one of examples herein, particularly example 105, wherein the one or more non-resorbable materials comprise polyester, co-polyesters, polyamide, polyolefin, polyurethanes, natural fibers, fluoropolymers, polyethers, polyureas, copolymers thereof, metal wires, or any combination thereof.

Example 107. The prosthetic valve of any one of examples herein, particularly example 105 or 106, wherein the at least one yarn of the plurality of weft yarns comprise a PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, cobalt-chromium (Co-Cr), nitinol (NiT), stainless steel, or a combination thereof.

Example 108. The prosthetic valve of any one of examples herein, particularly examples 84–107, wherein at least one yarn of the plurality of weft yarn is a twisted yarn with about 3 to about 16 turns per inch of yarn.

Example 109. The prosthetic valve of any one of examples herein, particularly examples 84–108, wherein at least one yarn of the plurality of weft yarns has a denier of about 10 D to about 50 D.

Example 110. The prosthetic valve of any one of examples herein, particularly examples 84–109, wherein at least one yarn of the plurality of weft yarns comprises about 1 filament to about 200 filaments.

Example 111. The prosthetic valve of any one of examples herein, particularly examples 84–110, wherein the at least one yarn of the plurality of yarns present in the cut pile layer is a flat yarn, a twisted yarn, a textured yarn, or a combination thereof.

Example 112. The prosthetic valve of any one of examples herein, particularly example 111, wherein the at least one yarn of the plurality of yarns present in the cut pile layer is the twisted yarn having 2 to 6 turns per inch of the yarn.

Example 113. The prosthetic valve of any one of examples herein, particularly examples 74–112, the at least one yarn of the plurality of yarns present in the cut pile layer comprises polyester, polyamide, polyolefin, fluoropolymer, or any combination thereof.

Example 114. The prosthetic valve of any one of examples herein, particularly example 113, wherein the at least one yarn of the plurality of yarns present in the cut pile layer comprises a PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, or a combination thereof.

Example 115. The prosthetic valve of any one of examples herein, particularly examples 74–114, wherein the at least one yarn of the plurality of yarns present in the cut pile layer has a denier of about 20 denier to about 200 denier.

Example 116. The prosthetic valve of any one of examples herein, particularly examples 74–115, wherein the at least one yarn of the plurality of yarns present in the cut pile layer has a filament count of about 1 to about 200 filaments.

Example 117. The prosthetic valve of any one of examples herein, particularly examples 74–116, wherein the first surface of the woven base layer comprises a layer of an elastomeric material or a low melt polymer.

Example 118. The prosthetic valve of any one of examples herein, particularly examples 74–117, wherein the covering has about 40 to about 400 ends per inch (EPI).

Example 119. The prosthetic valve of any one of examples herein, particularly examples 74–118, wherein the covering has about 40 to about 200 picks per inch (PPI).

Example 120. The prosthetic valve of any one of examples herein, particularly examples 74–119, wherein the covering exhibits a varying density.

Example 121. The prosthetic valve of any one of examples herein, particularly examples 74–120, wherein the predetermined thickness of the cut pile layer is about 0.1 mm to about 5 mm.

Example 122. A prosthetic valve comprising: at least one covering comprising: a double cloth material comprising a top portion and a bottom portion, wherein the top portion and bottom portion are spaced at a first length; wherein the top portion comprises a first woven base layer having a first surface and an opposing second surface and comprising a first plurality of warp yarns and a first plurality of weft yarns; wherein the bottom portion comprises a second woven base layer having a first surface and an opposing second surface comprising a second plurality of warp yarns and a second plurality of weft yarns; wherein the first and second woven base layers have a first predetermined pattern; wherein the top portion and bottom portion are coupled with a plurality of pile yarns such that the second surface of the first woven base layer faces the second surface of the second woven base layer, wherein the plurality of pile yarns extend between the second surface the first woven base layer and the second surface of the second woven base layer along the first length in a second predetermined pattern; wherein a first portion of the plurality of pile yarns is interwoven with the first woven base layer and a second portion of the plurality of pile yarns is interwoven with the second woven base layer; wherein the first woven base layer is bio-resorbable; wherein at least a portion of the second portion of the plurality of pile yarns is integrally fused with at least a portion of the second woven base layer; and wherein the at least one covering is positioned on a valve frame such that the second woven base layer is adjacent to the valve frame and the first woven base layer is exposed to a native anatomy.

Example 123. The prosthetic valve of any one of examples herein, particularly example 122, wherein the first woven base layer is configured to bio-resorb when exposed to the natural anatomy.

Example 124. The prosthetic valve of any one of examples herein, particularly example 122 or 123, wherein the first woven base layer comprises at least one plurality of yarns comprising one or more bio-resorbable materials.

Example 125. The prosthetic valve of any one of examples herein, particularly example 124, wherein the one or more bio-resorbable materials comprise PEO, PGA, PLLA, or a combination thereof.

Example 126. The prosthetic valve of any one of examples herein, particularly examples 122–125, wherein the first surface of the second woven base layer comprises a layer of an elastomeric material or a low melt polymer.

Example 127. The prosthetic valve of any one of examples herein, particularly examples 122–126, wherein the first predetermined pattern comprises a plain 1:1 pattern, warp rib 2/1, warp rib 2/2, plain weave, and its derivatives or any combination thereof.

Example 128. The prosthetic valve of any one of examples herein, particularly examples 125–127, wherein the first plurality of warp yarns comprise multifilament or monofilament yarns.

Example 129. The prosthetic valve of any one of examples herein, particularly examples 122–128, wherein the second plurality of warp yarns comprise multifilament or monofilament yarns.

Example 130. The prosthetic valve of any one of examples herein, particularly examples 128 or 129, wherein the first plurality of warp yarns comprises a number of filaments that is the same or different from the second plurality of warp yarns.

Example 131. The prosthetic valve of any one of examples herein, particularly examples 122–130, wherein the first plurality of warp yarns comprise one or more filaments having the same or different denier.

Example 132. The prosthetic valve of any one of examples herein, particularly examples 122–131, wherein the second plurality of warp yarns comprise one or more filaments having the same or different denier.

Example 133. The prosthetic valve of any one of examples herein, particularly examples 122–132, wherein at least a portion of the first plurality of warp yarns has the same or different denier as at least a portion of the second plurality of warp yarns.

Example 134. The prosthetic valve of any one of examples herein, particularly examples 122–133, wherein the first plurality and second plurality of weft yarn comprises a multifilament yarn.

Example 135. The prosthetic valve of any one of examples herein, particularly examples 122–134, wherein the first plurality and second plurality of weft yarn comprises a monofilament yarn.

Example 136. The prosthetic valve of any one of examples herein, particularly examples 122–135, wherein the second predetermined pattern of the pile yarn comprises a V-pattern, U-pattern, W-pattern, or a combination thereof.

Example 137. The prosthetic valve of any one of examples herein, particularly examples 122–136, wherein at least one yarn of the plurality of pile yarns is a multifilament yarn.

Example 138. The prosthetic valve of any one of examples herein, particularly example 137, wherein the covering is a cushioning covering.

Example 139. The prosthetic valve of any one of examples herein, particularly example 137 or 138, wherein the covering is configured to substantially reduce a PVL and/or increase tissue ingrowth.

Example 140. The prosthetic valve of any one of examples herein, particularly examples 122–139, wherein at least one yarn of the plurality of pile yarns is a monofilament yarn.

Example 141. The prosthetic valve of any one of examples herein, particularly example 140, wherein the covering is a frictional element configured to increase the retention of the valve in native anatomy or to couple various elements of the valve.

Example 142. The prosthetic valve of any one of examples herein, particularly examples 122–141, wherein the covering is positioned on an outer surface of the frame to form an outer skirt.

Example 143. The prosthetic valve of any one of examples herein, particularly examples 122–142, wherein the covering forms a covering of an outside valve component.

Example 144. The prosthetic valve of any one of examples herein, particularly example 143, wherein the outside valve component comprises one or more anchors present on the frame.

Example 145. The prosthetic valve of any one of examples herein, particularly examples 122–144, wherein at least one of the first and/or second plurality of warp yarns comprise a twisted yarn, a textured yarn, flat, or any combination thereof.

Example 146. The prosthetic valve of any one of examples herein, particularly examples 122–145, wherein at least one yarn in the second plurality of warp yarns comprises an elastomeric material.

Example 147. The prosthetic valve of any one of examples herein, particularly examples 122–146, wherein at least one yarn in the second plurality of warp yarns comprises at least one low melt polymer.

Example 148. The prosthetic valve of any one of examples herein, particularly examples 122–147, wherein at least one yarn in the second plurality of warp yarns comprises one or more of bi-component filaments.

Example 149. The prosthetic valve of any one of examples herein, particularly example 148, wherein the one or more of the bi-component filaments have core/sheath configuration, side-by-side configuration, lobal configuration, island-in- the sea configuration, segmented configuration, or any combination thereof.

Example 150. The prosthetic valve of any one of examples herein, particularly example 149, wherein the one or more bi-component filaments comprise the core/sheath configuration and wherein a sheath comprises a polymer with a lower melting temperature than a polymer in a core.

Example 151. The prosthetic valve of any one of examples herein, particularly examples 146–150, wherein the elastomeric material comprises one or more thermoplastic polyurethanes, elastomeric polyolefins, silicon-based materials, copolymers thereof, or any combination thereof.

Example 152. The prosthetic valve of any one of examples herein, particularly examples 122–151, wherein at least one yarn of the second plurality of warp yarns comprise one or more filaments comprising one or more non-resorbable materials.

Example 153. The prosthetic valve of any one of examples herein, particularly example 152, wherein the one or more non-resorbable materials comprise polyester, co-polyesters, polyamide, polyolefin, polyurethanes, natural fibers, fluoropolymers, polyethers, polyureas, copolymers thereof, metal wires, or any combination thereof.

Example 154. The prosthetic valve of any one of examples herein, particularly example 152 or 153, wherein the at least one yarn of the second plurality of warp yarns comprise a PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, cobalt-chromium (Co-Cr), nitinol (NiT), stainless steel, or a combination thereof.

Example 155. The prosthetic valve of any one of examples herein, particularly examples 122–154, wherein at least one yarn of the first and/or second plurality of warp yarn is a twisted yarn with about 3 to about 16 turns per inch of yarn.

Example 156. The prosthetic valve of any one of examples herein, particularly examples 122–155, wherein at least one yarn of the first and/or second plurality of warp yarns has a denier of about 15 D to about 50 D.

Example 157. The prosthetic valve of any one of examples herein, particularly examples 122–156, wherein at least one yarn of the first and/or second plurality of warp yarns comprises about 1 filament to about 200 filaments.

Example 158. The prosthetic valve of any one of examples herein, particularly examples 122–157, wherein at least one of the first and/or second plurality of weft yarns comprise a twisted yarn, a plied yarn, a textured yarn, flat, covered yarn, or any combination thereof.

Example 159. The prosthetic valve of any one of examples herein, particularly example 158, wherein at least one yarn in the second plurality of weft yarns comprises an elastomeric material.

Example 160. The prosthetic valve of any one of examples herein, particularly examples 122–159, wherein at least one yarn in the second plurality of weft yarns comprises at least one low melt polymer.

Example 161. The prosthetic valve of any one of examples herein, particularly examples 122–160, wherein at least one yarn in the second plurality of weft yarns comprises one or more of bi-component filament.

Example 162. The prosthetic valve of any one of examples herein, particularly example 161, wherein the one or more of the bi-component filaments have a core/sheath configuration, side-by-side configuration, lobal configuration, island-in- the sea configuration, segmented configuration, or any combination thereof.

Example 163. The prosthetic valve of any one of examples herein, particularly example 162, wherein the one or more bi-component filaments comprise the core/sheath configuration and wherein a sheath comprises a polymer with a lower melting temperature than a polymer in a core.

Example 164. The prosthetic valve of any one of examples herein, particularly examples 159–163, wherein the elastomeric material comprises one or more thermoplastic polyurethanes, elastomeric polyolefins, silicon-based materials, copolymers thereof, or any combination thereof.

Example 165. The prosthetic valve of any one of examples herein, particularly examples 122–164, wherein at least one yarn of the second plurality of weft yarns comprise one or more filaments comprising one or more non-resorbable materials.

Example 166. The prosthetic valve of any one of examples herein, particularly example 165, wherein the one or more non-resorbable materials comprise polyester, co-polyesters, polyamide, polyolefin, polyurethanes, natural fibers, fluoropolymers, polyethers, polyureas, copolymers thereof, metal wires, or any combination thereof.

Example 167. The prosthetic valve of any one of examples herein, particularly example 165 or 166, wherein the at least one yarn of the first and/or second plurality of weft yarns comprise a PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, cobalt-chromium (Co-Cr), nitinol (NiT), stainless steel, or a combination thereof.

Example 168. The prosthetic valve of any one of examples herein, particularly examples 122–167, wherein at least one yarn of the first and/or second plurality of weft yarn is a twisted yarn with about 3 to about 16 turns per inch of yarn.

Example 169. The prosthetic valve of any one of examples herein, particularly examples 122–168, wherein at least one yarn of the first and/or second plurality of weft yarns has a denier of about 10 D to about 50 D.

Example 170. The prosthetic valve of any one of examples herein, particularly examples 122–169, wherein at least one yarn of the first and/or second plurality of weft yarns comprises about 1 filament to about 200 filaments.

Example 171. The prosthetic valve of any one of examples herein, particularly examples 122–170, wherein at least one yarn of the plurality of pile yarns is a flat yarn, a twisted yarn, a textured yarn, or a combination thereof.

Example 172. The prosthetic valve of any one of examples herein, particularly example 171, wherein the at least one yarn of the plurality of pile yarns is the twisted yarn having 2 to 6 turns per inch of the yarn.

Example 173. The prosthetic valve of any one of examples herein, particularly example 171 or 172, wherein the at least one yarn of the plurality of pile yarns comprises polyester, polyamide, polyolefin, fluoropolymer, or any combination thereof.

Example 174. The prosthetic valve of any one of examples herein, particularly example 173, wherein the at least one yarn of the plurality of pile yarns comprises PET and co-polymers thereof, ultra-high molecular weight polyethylene (UHMWPE), polyethylene, polypropylene, polytetrafluoroethylene (PTFE), expanded polytetrafluorethylene (ePTFE), polyvinylidene fluoride, polyurethane, polyethers, polyureas, nylon, copolymers thereof, or a combination thereof.

Example 175. The prosthetic valve of any one of examples herein, particularly examples 122–174, wherein at least one yarn of the plurality of pile yarns has a denier of about 20 denier to about 200 denier.

Example 176. The prosthetic valve of any one of examples herein, particularly examples 122–175, wherein at least one yarn of the plurality of pile yarns has a filament count of about 1 to about 200.

Example 177. The prosthetic valve of any one of examples herein, particularly examples 122–176, wherein the first woven base layer and/or second woven base layer has about 40 to about 400 ends per inch (EPI).

Example 178. The prosthetic valve of any one of examples herein, particularly examples 122–177, wherein the first woven base layer and/or second woven base layer has about 40 to about 200 picks per inch (PPI).

Example 179. The prosthetic valve of any one of examples herein, particularly examples 122–178, wherein the first woven base layer and/or second woven base layer exhibits a varying density.

Example 180. The prosthetic valve of any one of examples herein, particularly examples 122–179, wherein the first predetermined length is up to 10 mm.

Although several aspects of the disclosure have been disclosed in the foregoing specification, it is understood by those skilled in the art that many modifications and other aspects of the disclosure will come to mind to which the disclosure pertains, having the benefit of the teaching presented in the foregoing description and associated drawings. Thus, it is understood that the disclosure is not limited to the specific aspects disclosed hereinabove and that many modifications and other aspects are intended to be included within the scope of the appended claims. Moreover, although specific terms are employed herein, as well as in the claims which follow, they are used only in a generic and descriptive sense and not for the purposes of limiting the described disclosure nor the claims which follow. Therefore, we claim as our disclosure all that comes within the scope and spirit of these claims.

We claim:

1. A method of forming an implantable prosthetic valve, wherein the method comprises:
(a) forming a valve covering material, wherein the step of forming comprises:
(i) weaving a double cloth comprising a top portion and a bottom portion, wherein the top portion and bottom portion are spaced at a first length;
wherein the top portion comprises a first woven base layer having a first surface and an opposing second surface and comprising a first plurality of warp yarns and a first plurality of weft yarns;
wherein the bottom portion comprises a second woven base layer having a first surface and an opposing second surface comprising a second plurality of warp yarns and a second plurality of weft yarns;
wherein the top portion and bottom portion are coupled with a plurality of pile yarns such that the second surface of the first woven base layer faces the second surface of the second woven base layer, wherein the plurality of pile yarns extend between the second surface of the first woven base layer and the second surface of the second woven base layer along the first length;
wherein a first portion of the plurality of pile yarns is interwoven with the first woven base layer, and a second portion of the plurality of pile yarns is interwoven with the second woven base layer;
(ii) cutting through the plurality of pile yarns transversely to the first length to form:
a first cloth material comprising the first woven base layer having a first predetermined pattern and a first cut pile layer comprising a portion of the plurality of pile yarns and extending outwardly from the second surface of the first woven base layer, wherein the first cut pile layer has a second predetermined pattern and a first predetermined thickness;
a second cloth material comprising the second woven base layer having the first predetermined pattern and a second cut pile layer comprising a portion of the plurality of pile yarns and extending outwardly from the second surface of the second woven base layer, wherein the second cut pile layer has the second predetermined pattern and a second predetermined thickness; and
wherein at least a portion of the first portion of the plurality of pile yarns is integrally fused with at least a portion of the first woven base layer; and/or
wherein at least a portion of the second portion of the plurality of pile yarns is integrally fused with at least a portion of the second woven base layer; and
(b) positioning at least one of the first and/or second cloth materials on a prosthetic valve frame to form the valve covering such that at least a portion of the first and/or second cut pile layer is exposed to a natural anatomy.

2. The method of claim 1,
wherein the first predetermined thickness is the same as the second predetermined thickness; or
wherein the first predetermined thickness is larger than the second predetermined thickness; or
wherein the first predetermined thickness is smaller than the second predetermined thickness.

3. The method of claim 1, wherein the first predetermined pattern of the first and/or the second woven base layer comprises a plain 1:1 pattern, warp rib 2/1, warp rib 2/2, plain weave, and its derivatives or any combination thereof.

4. The method of claim 1,
wherein the first plurality of warp yarns comprise multifilament or monofilament yarns, and/or
wherein the second plurality of warp yarns comprise multifilament or monofilament yarns.

5. The method of claim 1, wherein
the first plurality of warp yarns comprises a number of filaments that is the same or different from the second plurality of warp yarns; and/or
the first plurality of warp yarns comprise one or more filaments having the same or different denier; and/or
the second plurality of warp yarns comprise one or more filaments having the same or different denier; and/or
at least a portion of the first plurality of warp yarns has the same or different denier as at least a portion of the second plurality of warp yarns.

6. The method of claim 1, wherein
the first plurality of weft yarns and the second plurality of weft yarn comprise the same or different material, and/or
the first plurality and second plurality of weft yarn comprises a multifilament yarn; and/or
the first plurality and second plurality of weft yarn comprises a monofilament yarn.

7. The method of claim 1, wherein the second predetermined pattern of the first and/or the second cut pile layers comprises a V-pattern, U-pattern, W-pattern, or a combination thereof.

8. The method of claim 1, wherein at least one yarn of the plurality of pile yarns is a multifilament yarn, and wherein the covering is a cushioning covering.

9. The method of claim 1, wherein at least one yarn of the plurality of pile yarns is a monofilament yarn, and wherein the covering is a frictional element configured to increase the retention of the valve in native anatomy or to couple various elements of the valve.

10. The method of claim 1,
wherein the valve covering material is positioned on an outer surface of the prosthetic valve frame to form an outer skirt, and/or
wherein the valve covering material forms a covering of an outside valve component.

11. The method of claim 1, wherein
at least one of the first and/or second plurality of warp yarns comprise a twisted yarn, a textured yarn, flat, or any combination thereof; and/or
at least one yarn in the first or second plurality of warp yarns comprises an elastomeric material comprising one or more thermoplastic polyurethanes, elastomeric polyolefins, silicon-based materials, copolymers thereof, or any combination thereof; and/or
at least one yarn in the first or second plurality of warp yarns comprises at least one low melt polymer; and/or
at least one yarn in the first and/or second plurality of warp yarns comprises one or more bi-component filaments; and/or
at least one yarn of the first and/or second plurality of warp yarns comprise one or more filaments comprising one or more non-resorbable materials; and/or
at least one yarn of the first and/or second plurality of warp yarns comprise one or more filaments comprising one or more bio-resorbable materials; and/or
at least one yarn of the first and/or second plurality of warp yarn is a twisted yarn with about 3 to about 16 turns per inch of yarn; and/or
at least one yarn of the first and/or second plurality of warp yarns has a denier of about 15 D to about 50 D; and/or
at least one yarn of the first and/or second plurality of warp yarns comprises about 1 filament to about 200 filaments.

12. The method of claim 11, wherein the one or more bi-component filaments, if present, comprise a core/sheath configuration and wherein a sheath comprises a polymer with a lower melting temperature than a polymer in a core.

13. The method of claim 11, wherein the one or more non-resorbable materials, if present, comprise polyester, co-polyester, polyamide, polyolefin, polyurethanes, natural fibers, fluoropolymer, polyether, polyurea, copolymers thereof, metal wires, or any combination thereof.

14. The method of claim 11, wherein the one or more bio-resorbable materials comprise PEO, PGA, PLLA, or a combination thereof.

15. The method of claim 1, wherein
at least one of the first and/or second plurality of weft yarns comprise a twisted yarn, a plied yarn, a textured yarn, flat, covered yarn, or any combination thereof; and/or
at least one yarn in the first and/or second plurality of weft yarns comprises an elastomeric material; and/or
at least one yarn in the first or second plurality of weft yarns comprises at least one low melt polymer; and/or
at least one yarn in the first or second plurality of weft yarns comprises one or more bi-component filaments; and/or
at least one yarn of the first and/or second plurality of weft yarns comprise one or more filaments comprising one or more non-resorbable materials; and/or
at least one yarn of the first and/or second plurality of weft yarns comprise one or more filaments comprising one or more bio-resorbable materials; and/or
at least one yarn of the first and/or second plurality of weft yarn is a twisted yarn with about 3 to about 16 turns per inch of yarn; and/or
at least one yarn of the first and/or second plurality of weft yarns has a denier of about 10 D to about 50 D; and/or
at least one yarn of the first and/or second plurality of weft yarns comprises about 1 filament to about 200 filaments.

16. The method of claim 15, wherein the one or more bi-component filaments, if present, comprise a core/sheath configuration and wherein a sheath comprises a polymer with a lower melting temperature than a polymer in a core and/or wherein the elastomeric material, if present, comprises one or more thermoplastic polyurethanes, elastomeric polyolefins, silicon-based materials, copolymers thereof, or any combination thereof; and/or
wherein the one or more non-resorbable materials, if present, comprise polyester, co-polyester, polyamide, polyolefin, polyurethane, natural fiber, fluoropolymer, polyether, polyurea, copolymers thereof, metal wire, or any combination thereof; and/or
wherein the one or more bio-resorbable materials comprise PEO, PGA, PLLA, or a combination thereof.

17. The method of claim 1, wherein
at least one yarn of the plurality of pile yarns is a flat yarn, a twisted yarn, a textured yarn, or a combination thereof; and wherein the at least one yarn of the plurality of pile yarns is the twisted yarn having 2 to 6 turns per inch of the yarn; and/or
at least one yarn of the plurality of pile yarns comprises polyester, polyamide, polyolefin, fluoropolymer, metal wire, or any combination thereof; and/or
at least one yarn of the plurality of pile yarns has a denier of about 20 deniers to about 200 deniers; and/or
at least one yarn of the plurality of pile yarns has a filament count of about 1 to about 200.

18. The method of claim 1, wherein the first cloth and/or second cloth has about 40 to about 400 ends per inch (EPI); and/or wherein the first cloth and/or second cloth has about 40 to about 200 picks per inch (PPI), and/or
wherein the first and/or second cloth exhibits a varying density; and/or
wherein the first and/or second predetermined thicknesses of the first and/or second cut pile layer are about 0.1 mm to about 5 mm.

19. The method of claim 1, wherein the method comprises disposing a layer of an elastomeric material or a low melt polymer on the first surface of the first woven base layer; and/or
disposing a layer of an elastomeric material or a low melt polymer on the first surface of the second woven base layer.

20. The method of claim 19, heating the layer of the elastomeric material or the low melt polymer to integrally fuse at least a portion of the first portion of the plurality of pile yarns with at least a portion of the first woven base layer and/or at least a portion of the second portion of the plurality of pile yarns with at least a portion of the second woven base layer.

21. The method of claim 10, heating the first woven base layer to integrally fuse at least a portion of the first portion of the plurality of pile yarns with at least a portion of the first woven base layer and/or heating the second woven base layer to integrally fuse at least a portion of the second portion of the plurality of pile yarns with at least a portion of the second woven base layer.

22. The method of claim 20, wherein the heating comprises exposure to an IR heater, a UV heater, heater rollers, or a combination thereof and wherein the heating is performed during the weaving step and/or wherein the heating is performed during the cutting step; and/or
wherein the heating is performed after the cutting step.

23. A method of forming an implantable prosthetic valve, wherein the method comprises:
(a) forming a valve covering material, wherein the step of forming comprises:
weaving a double cloth comprising a top portion and a bottom portion, wherein the top portion and bottom portion are spaced at a first length;
wherein the top portion comprises a first woven base layer having a first surface and an opposing second surface and comprising a first plurality of warp yarns and a first plurality of weft yarns;
wherein the bottom portion comprises a second woven base layer having a first surface and an opposing second surface comprising a second plurality of warp yarns and a second plurality of weft yarns;
wherein the first and second woven base layers have a first predetermined pattern;
wherein the top portion and bottom portion are coupled with a plurality of pile yarns such that the second surface of the first woven base layer faces the second surface of the second woven base layer, wherein the plurality of pile yarns extend between the second surface of the first woven base layer and the second surface of the second woven base layer along the first length; wherein a first portion of the plurality of pile yarns is interwoven with the first woven base layer and a second portion of the plurality of pile yarns are interwoven with the second woven base layer;
wherein the first woven base layer is bio-resorbable;
wherein at least a portion of the second portion of the plurality of pile yarns is integrally fused with at least a portion of the second woven base layer; and
(b) positioning the covering on a prosthetic valve frame to form the valve covering such that the first surface of the first woven base layer is exposed to a natural anatomy.

24. A prosthetic valve comprising:
at least one covering comprising:
a woven base layer having a first surface and a second surface, and a first predetermined pattern;
a cut pile layer, wherein the cut pile layer has a predetermined thickness and a second predetermined pattern; and
wherein the cut pile layer comprises a plurality of yarns outwardly extending from the second surface of the woven base layer, wherein at least one yarn of the plurality of yarns present in the cut pile layer is a multifilament yarn or a monofilament yarn, and wherein at least a portion of the plurality of pile yarns is integrally fused with the woven base layer; and
wherein the at least one covering is positioned on a valve frame, such that the first surface of the woven base layer is adjacent to the frame.

25. A prosthetic valve comprising:
at least one covering comprising:
a double cloth material comprising a top portion and a bottom portion, wherein the top portion and bottom portion are spaced at a first length;
wherein the top portion comprises a first woven base layer having a first surface and an opposing second surface and comprising a first plurality of warp yarns and a first plurality of weft yarns;
wherein the bottom portion comprises a second woven base layer having a first surface and an opposing second surface comprising a second plurality of warp yarns and a second plurality of weft yarns;
wherein the first and second woven base layers have a first predetermined pattern;
wherein the top portion and bottom portion are coupled with a plurality of pile yarns such that the second surface of the first woven base layer faces the second surface of the second woven base layer, wherein the plurality of pile yarns extend between the second surface the first woven base layer and the second surface of the second woven base layer along the first length in a second predetermined pattern; wherein a first portion of the plurality of pile yarns is interwoven with the first woven base layer and a second portion of the plurality of pile yarns is interwoven with the second woven base layer;
wherein the first woven base layer is bio-resorbable;
wherein at least a portion of the second portion of the plurality of pile yarns is integrally fused with at least a portion of the second woven base layer; and
and wherein the at least one covering is positioned on a valve frame such that the second woven base layer is adjacent to the valve frame and the first woven base layer is exposed to a native anatomy.
